(19) United States
(12) Patent Application Publication
(10) Pub. No.: US 2021/0284659 A1
(43) Pub. Date: Sep. 16, 2021

(54) INHIBITORS OF BETA SECRETASE

(71) Applicant: Janssen Pharmaceutica NV, Beerse (BE)

(72) Inventors: Ann Marleen Vos, Boortmeerbeek (BE); Daniel Oehlrich, Geel (BE); Henricus Jacobus Maria Gijsen, Breda (NL); Karl Shawn Watts, Portland, OR (US); Sathesh Pangala Bhat, Jersey City, NJ (US); Peter Jacobus Johannes Antonius Buijnsters, Etten-Leur (NL); Sven Franciscus Anna Van Brandt, Beerse (BE); Jesús Manuel Alcázar-Vaca, Toledo (ES)

(21) Appl. No.: 16/489,856

(22) PCT Filed: Mar. 6, 2018
(86) PCT No.: PCT/EP2018/055402

§ 371 Date: Aug. 29, 2019

Related U.S. Application Data
(60) Provisional application No. 62/467,998, filed on Mar. 7, 2017.

(30) Foreign Application Priority Data
Sep. 7, 2017 (EP) 17189756.4

(51) Int. Cl. C07D 513/04

(52) U.S. Cl. CPC C07D 513/04

(57) ABSTRACT

The present invention relates to tricyclic inhibitors of beta-secretase having the structure shown in Formula (I) and (II)

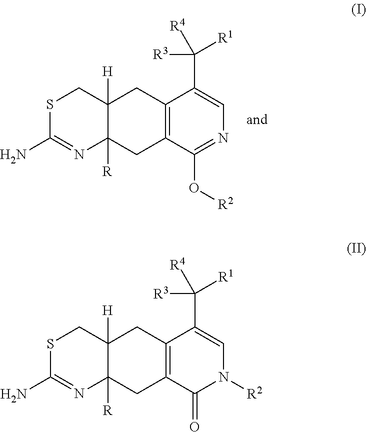

and the tautomers and the stereoisomeric forms thereof, wherein the radicals are as defined in the specification. The invention is also directed to pharmaceutical compositions comprising such compounds, to processes for preparing such compounds and compositions, and to the use of such compounds and compositions for the prevention and treatment of disorders in which beta-secretase is involved, such as Alzheimer's disease (AD), mild cognitive impairment, senility, dementia, dementia with Lewy bodies, Down's syndrome, dementia associated with stroke, dementia associated with Parkinson's disease, dementia associated with beta-amyloid, age-related macular degeneration, type 2 diabetes and other metabolic disorders.

### FIELD OF THE INVENTION

The present invention relates to tricyclic inhibitors of beta-secretase having the structure shown in Formula (I) and (II)

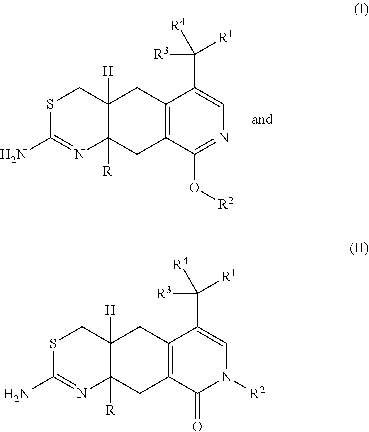

and the tautomers and the stereoisomeric forms thereof, wherein the radicals are as defined in the specification. The invention is also directed to pharmaceutical compositions comprising such compounds, to processes for preparing such compounds and compositions, and to the use of such compounds and compositions for the prevention and treatment of disorders in which beta-secretase is involved, such as Alzheimer's disease (AD), mild cognitive impairment, senility, dementia, dementia with Lewy bodies, Down's syndrome, dementia associated with stroke, dementia associated with Parkinson's disease, dementia associated with beta-amyloid, age-related macular degeneration, type 2 diabetes and other metabolic disorders.

### BACKGROUND OF THE INVENTION

Alzheimer's Disease (AD) is a neurodegenerative disease associated with aging. AD patients suffer from cognition deficits and memory loss as well as behavioral problems such as anxiety. Over 90% of those afflicted with AD have a sporadic form of the disorder while less than 10% of the cases are familial or hereditary. In the United States, about one in ten people at age 65 have AD while at age 85, one out of every two individuals are afflicted by AD. The average life expectancy from the initial diagnosis is 7-10 years, and AD patients require extensive care either in an assisted living facility or by family members. With the increasing number of elderly in the population, AD is a growing medical concern. Currently available therapies for AD merely treat the symptoms of the disease and include acetylcholinesterase inhibitors to improve cognitive properties as well as anxiolytics and antipsychotics to control the behavioral problems associated with this ailment.

The hallmark pathological features in the brain of AD patients are neurofibrillary tangles which are generated by hyperphosphorylation of tau protein and amyloid plaques which form by aggregation of beta-amyloid 1-42 (Abeta 1-42) peptide. Abeta 1-42 forms oligomers and then fibrils, and ultimately amyloid plaques. The oligomers and fibrils are believed to be especially neurotoxic and may cause most of the neurological damage associated with AD. Agents that prevent the formation of Abeta 1-42 have the potential to be disease-modifying agents for the treatment of AD. Abeta 1-42 is generated from the amyloid precursor protein (APP), comprised of 770 amino acids. The N-terminus of Abeta 1-42 is cleaved by beta-secretase (BACE1), and then gamma-secretase cleaves the C-terminal end. In addition to Abeta 1-42, gamma-secretase also liberates Abeta 1-40 which is the predominant cleavage product as well as Abeta 1-38 and Abeta 1-43. These Abeta forms can also aggregate to form oligomers and fibrils. Thus, inhibitors of BACE1 would be expected to prevent the formation of Abeta 1-42 as well as Abeta 1-40, Abeta 1-38 and Abeta 1-43 and would be potential therapeutic agents in the treatment of AD.

Type 2 diabetes (T2D) is caused by insulin resistance and inadequate insulin secretion from pancreatic beta-cells leading to poor blood-glucose control and hyperglycemia. Patients with T2D have an increased risk of microvascular and macrovascular disease and a range of related complications including diabetic nephropathy, retinopathy and cardiovascular disease. The rise in prevalence of T2D is associated with an increasingly sedentary lifestyle and high-energy food intake of the world's population.

Beta-cell failure and consequent dramatic decline in insulin secretion and hyperglycemia marks the onset of T2D. Most current treatments do not prevent the loss of beta-cell mass characterizing overt T2D. However, recent developments with GLP-1 analogues, gastrin and other agents show that preservation and proliferation of beta-cells is possible to achieve, leading to an improved glucose tolerance and slower progression to overt T2D.

Tmem27 has been identified as a protein promoting beta-cell proliferation and insulin secretion. Tmem27 is a 42 kDa membrane glycoprotein which is constitutively shed from the surface of beta-cells, resulting from a degradation of the full-length cellular Tmem27. Overexpression of Tmem27 in a transgenic mouse increases beta-cell mass and improves glucose tolerance in a diet-induced obesity DIO model of diabetes. Furthermore, siRNA knockout of Tmem27 in a rodent beta-cell proliferation assay (e.g. using INS1e cells) reduces the proliferation rate, indicating a role for Tmem27 in control of beta-cell mass.

BACE2 is the protease responsible for the degradation of Tmem27. It is a membrane-bound aspartyl protease and is co-localized with Tmem27 in human pancreatic beta-cells. It is also known to be capable of degrading APP, IL-1R2 and ACE2. The capability to degrade ACE2 indicates a possible role of BACE2 in the control of hypertension.

Inhibitors of BACE1 and/or BACE2 may in addition be used for the therapeutic and/or prophylactic treatment of amyotrophic lateral sclerosis (ALS), arterial thrombosis, autoimmune/inflammatory diseases, cancer such as breast cancer, cardiovascular diseases such as myocardial infarction and stroke, dermatomyositis, Down's Syndrome, gastrointestinal diseases, Glioblastoma multiforme, Graves Disease, Huntington's Disease, inclusion body myositis (IBM), inflammatory reactions, Kaposi Sarcoma, Kostmann Disease, lupus erythematosus, macrophagic myofasciitis, juvenile idiopathic arthritis, granulomatous arthritis, malignant melanoma, multiple myeloma, rheumatoid arthritis, Sjogren syndrome, SpinoCerebellar Ataxia 1, SpinoCerebellar Ataxia 7, Whipple's Disease or Wilson's Disease.

### SUMMARY OF THE INVENTION

The present invention is directed to compounds of Formula (I) and (II)

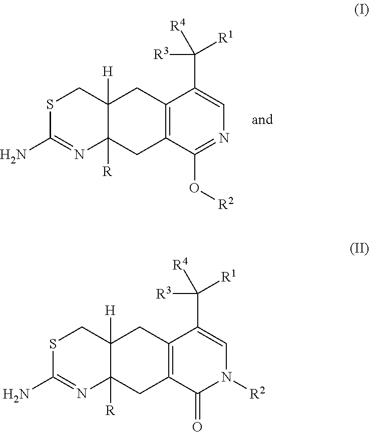

and the tautomers and the stereoisomeric forms thereof, wherein

R is phenyl optionally substituted with 1, 2, or 3 substituents each independently selected from the group consisting of halo, C₁₋₃alkyloxy, cyano, 2-cyano-pyridin-5-yl, 3-cyano-pyridin-5-yl, and pyrimidin-5-yl;

R¹ is selected from the group consisting of C₁₋₃alkyl; C₃₋₆cycloalkyl optionally substituted with C₁₋₃alkyl; aryl; heteroaryl; and 4-tetrahydro-2H-pyranyl optionally substituted with C₁₋₃alkyl; with the provisos that

a) R¹ is C₃₋₆cycloalkyl optionally substituted with C₁₋₃alkyl; aryl; heteroaryl; or 4-tetrahydro-2H-pyranyl optionally substituted with C₁₋₃alkyl; when R³ is hydrogen and R⁴ is hydrogen or C₁₋₃alkyl; or
b) R¹ is C₁₋₃alkyl; C₃₋₆cycloalkyl optionally substituted with C₁₋₃alkyl; aryl; heteroaryl; or 4-tetrahydro-2H-pyranyl optionally substituted with C₁₋₃alkyl; when R³ is hydrogen and R⁴ is C₁₋₃alkyloxy; or
c) R¹ is C₃₋₆cycloalkyl optionally substituted with C₁₋₃alkyl; or 4-tetrahydro-2H-pyranyl optionally substituted with C₁₋₃alkyl; when R³ is hydrogen and R⁴ is C₃₋₆cycloalkyl; or
d) R¹ is C₁₋₃alkyl; C₃₋₆cycloalkyl optionally substituted with C₁₋₃alkyl; aryl; heteroaryl; or 4-tetrahydro-2H-pyranyl optionally substituted with C₁₋₃alkyl; when >CR³R⁴ is >(C═O); wherein

aryl is phenyl or phenyl substituted with 1, 2 or 3 substituents each independently selected from the group consisting of halo, cyano, C₁₋₃alkyl, mono-halo-C₁₋₃alkyl, poly-halo-C₁₋₃alkyl, C₃₋₆cycloalkyl, C₁₋₃alkyloxy, mono-halo-C₁₋₃alkyloxy and polyhalo-C₁₋₃alkyloxy;

heteroaryl is selected from the group consisting of pyridyl, pyrimidyl, pyrazinyl, pyridazinyl, furanyl, thienyl, pyrrolyl, pyrazolyl, imidazolyl, triazolyl, tetrazolyl, thiazolyl, isothiazolyl, thiadiazolyl, oxazolyl, isoxazolyl, oxadiazolyl, indolyl, indazolyl, 1H-benzimidazolyl, benzoxazolyl, and benzothiazolyl, each of which being optionally substituted with 1, 2, or 3 substituents, each independently selected from the group consisting of halo, cyano, C₁₋₃alkyl, mono-halo-C₁₋₃alkyl, poly-halo-C₁₋₃alkyl, C₃₋₆ cycloalkyl, C₁₋₃alkyloxy, mono-halo-C₁₋₃alkyloxy and polyhalo-C₁₋₃alkyloxy; and

R² is hydrogen or C₁₋₃alkyl;
and the pharmaceutically acceptable addition salts and the solvates thereof.

Illustrative of the invention is a pharmaceutical composition comprising a pharmaceutically acceptable carrier and any of the compounds described above. An illustration of the invention is a pharmaceutical composition made by mixing any of the compounds described above and a pharmaceutically acceptable carrier. Illustrating the invention is a process for making a pharmaceutical composition comprising mixing any of the compounds described above and a pharmaceutically acceptable carrier.

Exemplifying the invention are methods of treating a disorder mediated by the beta-secretase enzyme, comprising administering to a subject in need thereof a therapeutically effective amount of any of the compounds or pharmaceutical compositions described above.

Further exemplifying the invention are methods of inhibiting the beta-secretase enzyme, comprising administering to a subject in need thereof a therapeutically effective amount of any of the compounds or pharmaceutical compositions described above.

An example of the invention is a method of treating a disorder selected from the group consisting of Alzheimer's disease, mild cognitive impairment, senility, dementia, dementia with Lewy bodies, Down's syndrome, dementia associated with stroke, dementia associated with Parkinson's disease, dementia associated with beta-amyloid, and age-related macular degeneration, preferably Alzheimer's disease, type 2 diabetes and other metabolic disorders, comprising administering to a subject in need thereof, a therapeutically effective amount of any of the compounds or pharmaceutical compositions described above.

Another example of the invention is any of the compounds described above for use in treating: (a) Alzheimer's Disease, (b) mild cognitive impairment, (c) senility, (d) dementia, (e) dementia with Lewy bodies, (f) Down's syndrome, (g) dementia associated with stroke, (h) dementia associated with Parkinson's disease, (i) dementia associated with beta-amyloid or (j) age-related macular degeneration, (k) type 2 diabetes and (1) other metabolic disorders in a subject in need thereof.

### DETAILED DESCRIPTION OF THE INVENTION

The present invention is directed to compounds of Formula (I) and (II) as defined hereinbefore, and pharmaceutically acceptable addition salts and solvates thereof. The compounds of formula (I) and (II) are inhibitors of the beta-secretase enzyme (also known as beta-site cleaving enzyme, BACE, BACE1, Asp2 or memapsin 2, or BACE2), and may be useful in the treatment of Alzheimer's disease, mild cognitive impairment, senility, dementia, dementia associated with stroke, dementia with Lewy bodies, Down's syndrome, dementia associated with Parkinson's disease, dementia associated with beta-amyloid, and age-related macular degeneration, preferably Alzheimer's disease, mild cognitive impairment or dementia, more preferably Alzheimer's disease, type 2 diabetes and other metabolic disorders.

In an embodiment, the present invention relates to compounds of Formula (I) and (II), as described herein, wherein

R¹ is C₃₋₆cycloalkyl optionally substituted with C₁₋₃alkyl; aryl; heteroaryl; or 4-tetrahydro-2H-pyranyl optionally substituted with C₁₋₃alkyl;
R³ is hydrogen;
R⁴ is hydrogen or C₁₋₃alkyl; wherein

aryl is phenyl or phenyl substituted with 1, 2 or 3 substituents each independently selected from the group consisting of halo, cyano, C₁₋₃alkyl, mono-halo-C₁₋₃alkyl, poly-halo-C₁₋₃alkyl, C₃₋₆cycloalkyl, C₁₋₃alkyloxy, mono-halo-C₁₋₃alkyloxy and polyhalo-C₁₋₃alkyloxy; and

heteroaryl is selected from the group consisting of pyridyl, pyrimidyl, pyrazinyl, pyridazinyl, furanyl, thienyl, pyrrolyl, pyrazolyl, imidazolyl, triazolyl, tetrazolyl, thiazolyl, isothiazolyl, thiadiazolyl, oxazolyl, isoxazolyl, oxadiazolyl, indolyl, indazolyl, 1H-benzimidazolyl, benzoxazolyl, and benzothiazolyl, each of which being optionally substituted with 1, 2, or 3 substituents, each independently selected from the group consisting of halo, cyano, C₁₋₃alkyl, mono-halo-C₁₋₃alkyl, poly-halo-C₁₋₃alkyl, C₃₋₆cycloalkyl, C₁₋₃alkyloxy, mono-halo-C₁₋₃alkyloxy and polyhalo-C₁₋₃alkyloxy;

and R² is as defined herein.

In another embodiment, the present invention relates to compounds of Formula (I) and (II), as described herein, wherein

aryl is phenyl or phenyl substituted with 1, 2 or 3 substituents each independently selected from the group consisting of of halo, cyano, C₁₋₃alkyl, mono-halo-C₁₋₃alkyl, poly-halo-C₁₋₃alkyl, C₃₋₆cycloalkyl, and C₁₋₃alkyloxy; and

heteroaryl is selected from the group consisting of pyridyl, pyrimidyl, pyrazinyl, pyridazinyl, furanyl, thienyl, pyrrolyl, pyrazolyl, imidazolyl, triazolyl, tetrazolyl, thiazolyl, isothiazolyl, thiadiazolyl, oxazolyl, isoxazolyl, and oxadiazolyl, each of which being optionally substituted with 1, 2, or 3 substituents, each independently selected from the group consisting of halo, cyano, C₁₋₃alkyl, mono-halo-C₁₋₃alkyl, poly-halo-C₁₋₃alkyl, C₃₋₆cycloalkyl, and C₁₋₃alkyloxy;

and R, R², R³, and R⁴ are as defined herein.

In an embodiment, the present invention relates to compounds of Formula (I) and (II), as described herein, wherein

R¹ is aryl; heteroaryl; or 4-tetrahydro-2H-pyranyl optionally substituted with C₁₋₃alkyl; R³ and R⁴ are each hydrogen;
aryl is phenyl or phenyl substituted with 1, 2 or 3 substituents each independently selected from the group consisting of of halo, C₁₋₃alkyl, and C₁₋₃alkyloxy; and
heteroaryl is selected from the group consisting of pyridyl, pyrimidyl, pyrazinyl, pyridazinyl, furanyl, thienyl, pyrrolyl, pyrazolyl, imidazolyl, triazolyl, tetrazolyl, thiazolyl, isothiazolyl, thiadiazolyl, oxazolyl, isoxazolyl, and oxadiazolyl, each of which being optionally substituted with 1, 2, or 3 substituents, each independently selected from the group consisting of halo, C₁₋₃alkyl, and C₁₋₃alkyloxy;
and R and R² are as defined herein.

In an embodiment, the present invention relates to compounds of Formula (I) and (II), as described herein, wherein

R is phenyl optionally substituted with 1, or 2 independently selected halo substituents; and R¹-R⁴ are as defined herein.

In a further embodiment, the present invention relates to compounds of Formula (I) and (II), as described herein, wherein

R² is C₁₋₃alkyl, in particular, methyl, and R, R¹, R³ and R⁴ are as defined herein.

The invention relates in particular to compounds wherein carbon centres C₄ₐ and C₁₀ₐ in the tricyclic scaffold are of cis configuration (i.e. H and R are projected towards the same side out of the plane of the scaffold)

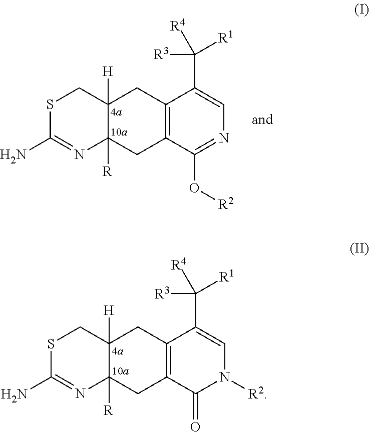

Thus, in particular, the invention relates to compounds of Formula (I′) and (II′) and compounds of Formula (I″) and (II″) as represented below, wherein the tricyclic core is in the plane of the drawing and H and R are projected above the plane of the drawing (with the bond shown with a bold wedge [symbol] ) in (I′) and (II′) or wherein the tricyclic core is in the plane of the drawing and H and R are projected below the plane of the drawing (with the bond shown with a wedge of parallel lines [symbol] ) in (I″) and (II″):

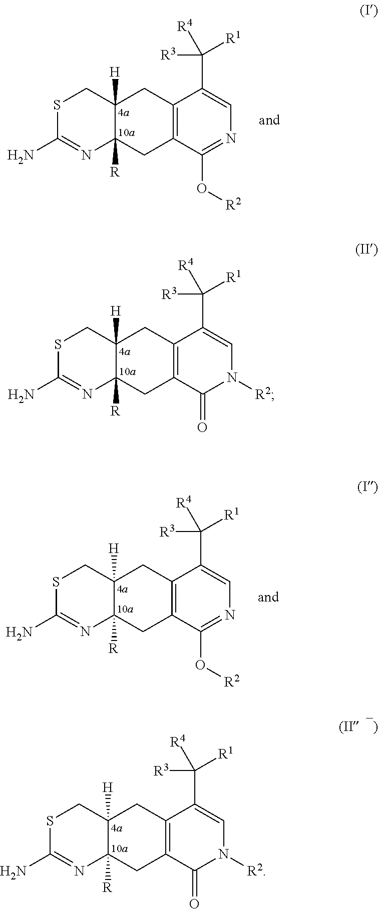

### Definitions

“Halo” shall denote fluoro, chloro and bromo; “C₁₋₃alkyl” shall denote a straight or branched saturated alkyl group having 1, 2 or 3 carbon atoms, e.g. methyl, ethyl, 1-propyl, 2-propyl, etc.; “C₁₋₃alkyloxy” shall denote an ether radical wherein C₁₋₃alkyl is as defined before; “mono- and polyhaloC₁₋₃alkyl” shall denote C₁₋₃alkyl as defined before, substituted with 1, 2, 3 or where possible with more halo atoms as defined before; “mono- and polyhaloC₁₋₃alkyloxy” shall denote an ether radical wherein mono- and polyhaloC₁₋₃alkyl is as defined before; “C₃₋₆cycloalkyl” shall denote cyclopropyl, cyclobutyl, cyclopentyl and cyclohexyl.

The term “subject” as used herein, refers to an animal, preferably a mammal, most preferably a human, who is or has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

Hereinbefore and hereinafter, the term “compound of formula (I) and (II)” is meant to include the addition salts, the solvates and the stereoisomers thereof.

The terms “stereoisomers” or “stereochemically isomeric forms” hereinbefore or hereinafter are used interchangeably.

The invention includes all stereoisomers of the compound of Formula (I) and (II) either as a pure stereoisomer or as a mixture of two or more stereoisomers.

Enantiomers are stereoisomers that are non-superimposable mirror images of each other. A 1:1 mixture of a pair of enantiomers is a racemate or racemic mixture. Diastereomers (or diastereoisomers) are stereoisomers that are not enantiomers, i.e. they are not related as mirror images. If a compound contains a double bond, the substituents may be in the E or the Z configuration. If a compound contains a disubstituted cycloalkyl group, the substituents may be in the cis or trans configuration. Therefore, the invention includes enantiomers, diastereomers, racemates, E isomers, Z isomers, cis isomers, trans isomers and mixtures thereof.

The absolute configuration is specified according to the Cahn-Ingold-Prelog system. The configuration at an asymmetric atom is specified by either R or S. Resolved compounds whose absolute configuration is not known can be designated by (+) or (−) depending on the direction in which they rotate plane polarized light.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other isomers. Thus, when a compound of formula (I) or (II) is for instance specified as (R), this means that the compound is substantially free of the (S) isomer; when a compound of formula (I) or (II) is for instance specified as E, this means that the compound is substantially free of the Z isomer; when a compound of formula (I) or (II) is for instance specified as cis, this means that the compound is substantially free of the trans isomer.

For use in medicine, the addition salts of the compounds of this invention refer to non-toxic “pharmaceutically acceptable addition salts”. Other salts may, however, be useful in the preparation of compounds according to this invention or of their pharmaceutically acceptable addition salts. Suitable pharmaceutically acceptable addition salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable addition salts thereof may include alkali metal salts, e.g., sodium or potassium salts; alkaline earth metal salts, e.g., calcium or magnesium salts; and salts formed with suitable organic ligands, e.g., quaternary ammonium salts.

Representative acids which may be used in the preparation of pharmaceutically acceptable addition salts include, but are not limited to, the following: acetic acid, 2,2-dichloroactic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, capric acid, caproic acid, caprylic acid, cinnamic acid, citric acid, cyclamic acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, beta-oxo-glutaric acid, glycolic acid, hippuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebacic acid, stearic acid, succinic acid, sulfuric acid, tannic acid, (+)-L-tartaric acid, thiocyanic acid,

p-toluenesulfonic acid, trifluoromethylsulfonic acid, and undecylenic acid. Representative bases which may be used in the preparation of pharmaceutically acceptable addition salts include, but are not limited to, the following: ammonia, L-arginine, benethamine, benzathine, calcium hydroxide, choline, dimethylethanolamine, diethanolamine, diethylamine, 2-(diethylamino)-ethanol, ethanolamine, ethylene-diamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide. A particular salt is the trifluoroacetic acid addition salt.

The names of compounds were generated according to the nomenclature rules agreed upon by the Chemical Abstracts Service (CAS) or according to the nomenclature rules agreed upon by the International Union of Pure and Applied Chemistry (IUPAC). In case of tautomeric forms, the name of the depicted tautomeric form of the structure was generated. The other non-depicted tautomeric form is also included within the scope of the present invention.

### Preparation of the Compounds

### Experimental Procedure 1

Final compounds according to Formula (I) and (II) can be prepared from intermediate compounds of Formula (III) and (IV) by Negishi type reactions according to art-known reaction conditions. Such conditions typically involve the reaction of intermediates of Formula (III) and (IV) with an organozinc compound of Formula (V) in the presence of a Palladium(0), e.g. Pd(PPh₃)₄, or a Nickel catalyst, a ligand, e.g. RuPhos, triphenylphosphine, dppe, BINAP. In Reaction scheme 1, X represents halo or triflate, X′ is halo and all other variables are as defined in Formula (I) and (II).

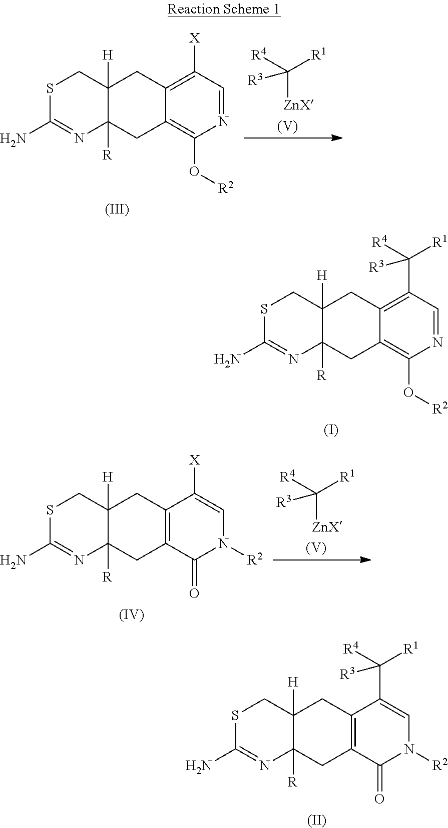

### Preparation of the Intermediate Compounds

### Experimental Procedure 2

Intermediate compounds of Formula (III) and (IV) can be obtained by deprotecting intermediate compounds of Formula (VI) and (VII) wherein Q and PG represent a base labile (e.g. an acyl) or acid labile (e.g. trityl) protecting group. In Reaction Scheme 2, X represents a halo or a triflate group, in particular bromo and all other variables are as defined in Formula (I) and (II).

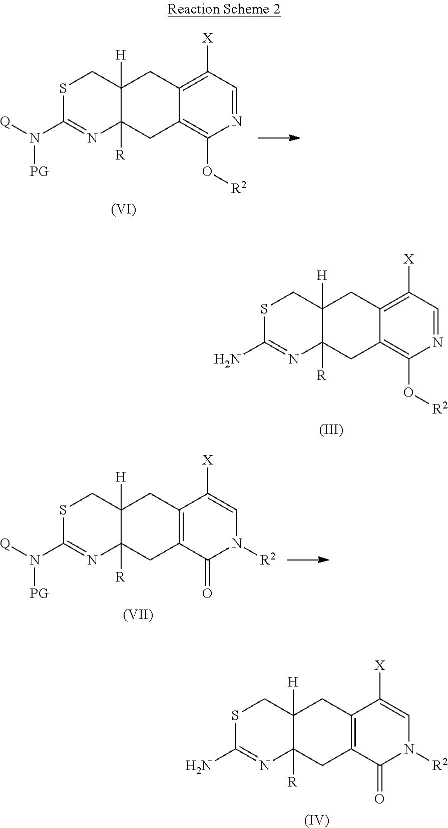

### Experimental Procedure 3

Intermediate compounds of Formula (III) wherein X is Br, herein referred to as intermediates of Formula (III-a) can be prepared from an intermediate compound of Formula (III-b) by art-known bromination procedures. Said bromination may conveniently be conducted by treatment of the corresponding intermediate compounds of Formula (III-b) with a brominating agent such as, for example, N-bromosuccinimide in a suitable inert solvent such as, for example, acetonitrile and the like at a suitable temperature such as, for example, room temperature (r.t.), until completion of the reaction, for example 16 hours.

Intermediates compound of Formula (III-b) may need to be protected by a protecting group PG such as, for example, tert-butoxycarbonyl group, following art-known procedures. Said reaction can conveniently be conducted by treatment of intermediate compound (III-b) with di-tert-butyl dicarbonate, in the presence of a suitable catalyst, such as, 4-(dimethylamino)pyridine (DMAP), in a suitable inert solvent such as, THF, under suitable reaction conditions, such as at a convenient temperature, typically r.t., for a period of time to ensure the completion of the reaction.

The protected intermediate (III-c) may then be brominated as described above to yield (III-d) which than may be deprotected by treatment with a suitable acid, such as for example, trifluoroacetic acid of formic acid in a suitable solvent, or neat, at ambient temperature to yield intermediate (III-a).

In Reaction Scheme 3, Q and PG are protecting groups and all other variables are defined as in Formula (I).

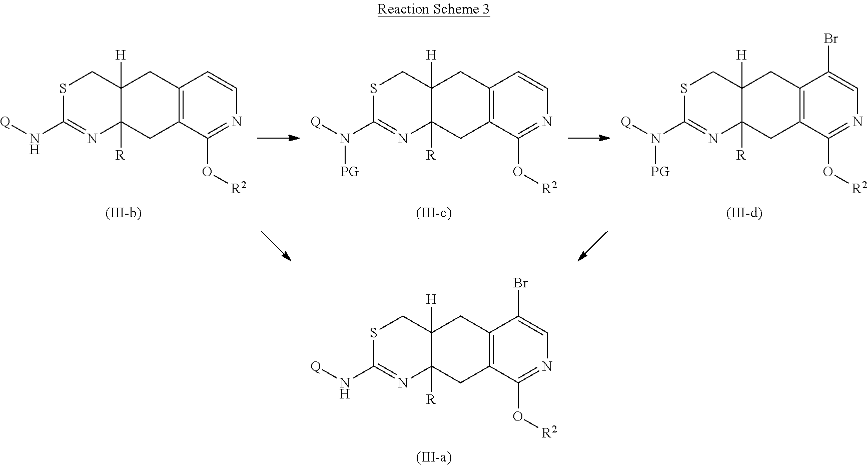

Alternatively, an Intermediate of Formula (III-a) can be deprotected under analogous procedures to those of Reaction Scheme 2.

### Experimental Procedure 4

Intermediates of Formula (III) wherein R² is C₁₋₃alkyl, herein referred to as intermediates of Formula (III-e) can be prepared by reaction the corresponding intermediates of Formula (III-b) wherein R² is methyl, herein referred to as intermediates of Formula (III-b′) with C₁₋₃alkyl iodide (Reaction Scheme 3). The reaction can be performed under thermal conditions such as, for example, heating the reaction mixture at 100° C. In Reaction Scheme 4, all variables are defined as in Formula (I).

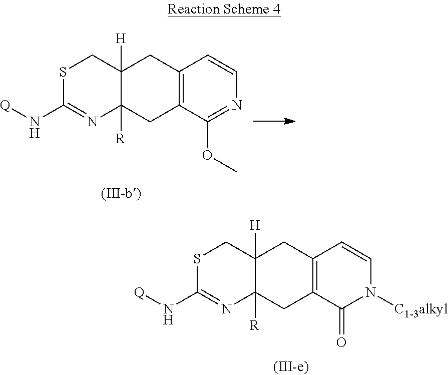

### Experimental Procedure 5

Intermediate compounds of Formula (III-b) can be prepared from an intermediate compound of Formula (VIII) following art-known cyclization procedures. Said cyclization may conveniently be conducted by treatment of an intermediate compound of Formula (VIII) with a suitable reagent, such as 1-chloro-N,N-2-trimethylpropenylamine, in a suitable reaction solvent, such as for example DCM under suitable reaction conditions, such as at a convenient temperature, typically r.t., for a period of time to ensure the completion of the reaction.

Intermediate compounds of Formula (VIII) can be prepared by reacting the corresponding intermediate compounds of Formula (IX) with a suitable reagent, such as, benzyl isothiocyanate (resulting in compounds (VIII) and (III-d) wherein Q is phenyl(C═O)—), in a suitable inert solvent, such as, for example, DCM, at a convenient temperature, typically r.t., until completion of the reaction, for example 3 hours.

Intermediate compounds of Formula (IX) can be prepared from the corresponding intermediate compounds of Formula (X) following art-known aziridine ring opening procedures. Said reaction may be carried out by stirring the reactants under a hydrogen atmosphere in the presence of an appropriate catalyst such as, for example, Raney-nickel in a suitable solvent, such as, for example, alkanols, e.g. methanol, ethanol and the like, at a convenient temperature, typically r.t., until completion of the reaction, for example 6 hours.

Intermediate compounds of Formula (X) can be prepared by reacting the corresponding intermediate compounds of Formula (XI) with an intermediate of Formula (XII), wherein R is as previously defined and X is for example, —Mg-halide. The reaction can be performed in a suitable reaction inert solvent, such as, THF under suitable reaction conditions, such as at a suitable temperature, typically in a range between −78° C. and room temperature, for a period of time to ensure the completion of the reaction. An intermediate compound of Formula (XII) can be obtained commercially or synthesized according to literature procedures.

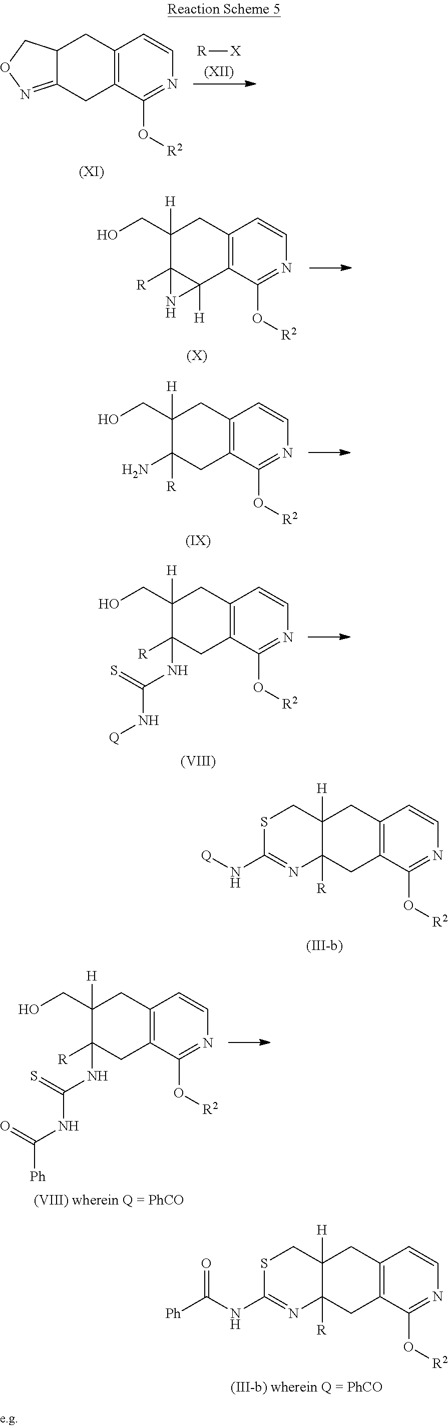

### Experimental Procedure 6

Intermediate compounds of Formula (XI) can be prepared by reacting the corresponding intermediate compounds of Formula (XIII) following art-known cyclization procedures. Said cyclization may be conveniently conducted by treatment of an intermediate compound of Formula (XIII) with a suitable acid, such as, for example hydrochloric acid, in a suitable reaction inert solvent, such as, THF under suitable reaction conditions, such as at a suitable temperature, typically 50° C., for a period of time to ensure the completion of the reaction.

Intermediate compounds of Formula (XIII) can be prepared by reacting the intermediate compounds of Formula (XIV) following art-known coupling procedures. Said transformation may be conveniently conducted by conversion of an intermediate compound of Formula (XIV) to the corresponding cyanocuprate reagent in the presence of a suitable metalation reagent, such as, isopropylmagnesium chloride lithium chloride complex, and a suitable organocuprate precursor, such as, for example, copper(I) cyanide di(lithium chloride) complex solution, followed by addition of a suitable halide, such as allyl bromide. Reaction may be performed in a suitable inert solvent, such as, for example, THF and the like solvents, at a convenient temperature, typically −70° C.-r.t. for a period of time to ensure the completion of the reaction.

Intermediate compounds of Formula (XIV) can be prepared by reacting the intermediate compounds of Formula (XV) following art-known Wittig reaction procedures. Said reaction may conveniently be conducted by treatment of the intermediate compound of Formula (XV) with a suitable phosphonium salt, such as, for example, methoxymethyl triphenylphosphonium chloride, in the presence of a suitable base such as, for example, potassium bis(trimethylsilyl)amide, in a suitable reaction-inert solvent, such as, for example, toluene, at convenient temperature, typically −10° C.-r.t., for a period of time to ensure the completion of the reaction.

Intermediate compounds of Formula (XV) can generally be obtained commercially or synthesized according to literature procedures.

In Reaction Scheme 6, all variables are defined as in Formula (I).

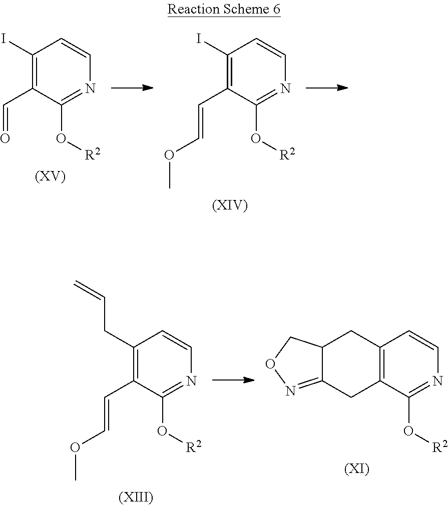

### Experimental Procedure 7

Alternatively, intermediate compounds of Formula (XI) can undergo addition of an organometallic species of Formula (XII-a), where R′ is any radical which can be converted into R by using procedures known to the person skilled in the art, such as, for example, cross coupling reactions, alkylation reactions and deprotection reactions. Intermediate compounds (X-a) can be carried on in the synthesis using the same synthetic pathway described in the examples before. The person skilled in the art will be able to judge at which point of the synthetic sequence the conversion of R′ to R is appropriate to perform.

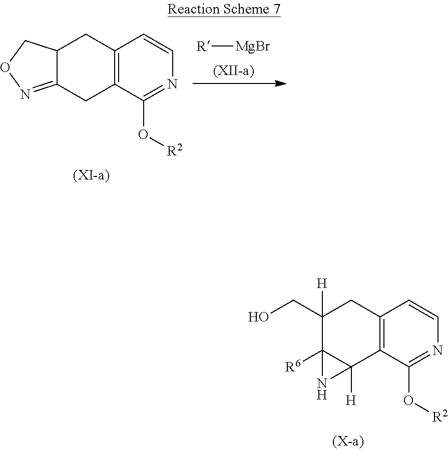

### Preparation of the Compounds—Flow Chemistry

A number of compounds were synthesized and screened using the CyclOps™ platform as described herein, which worked with a high success range (61-96% success rate). The flow synthesis system utilized the Vapourtec® R4 reactors and R2 pump modules with integrated valves and reagent loops controlled by FlowCommander™ software. Up to four reactors, pumps and valves were used depending on the complexity of the chemistry. The output from the final reactor flowed into a HPLC injection valve enabling an aliquot of product to be injected onto the purification system. Loss of material due to dispersion in the synthesis system was minimized in several ways. Firstly small bore tubing was used throughout the system as this minimised dispersion. Secondly, the reagent loop sizes were selected to ensure a steady state concentration of reactants and product was achieved in the reactor. Finally, the injection to HPLC was timed to ensure that an aliquot was taken at the point of maximum product concentration, i.e. under steady state conditions. In general, the use of fresh bottles of reagents and/or generating reagents in situ may improve the synthetic outcome.

### Experimental Procedure 8

Final compounds according to Formula (I) and (II) can be prepared from intermediate compounds of Formula (III) and (IV) by means of Negishi type reactions. Typically, intermediate of Formula (III-a′) is placed in one vessel in solvent (e.g. NMP). In a second vessel a bromo or chloro Negishi zincate (e.g. benzylzinc(II) bromide) is added and in a third vessel the catalyst in a suitable solvent (e.g. Pd(dppf)Cl₂ or RuPhos-Pd-G2 in NME, or THF) is prepared. The three vessels are loaded onto a Gilson 215 and injected into 250 injection loops and subsequently onto a 2 mL stainless steel coil heated to 80° C. with each pump running at 33 μL/min. Completion of the reaction occurs typically in 20 min. The outflow injects automatically through a 20 μL loop into the purification and assay part of the platform.

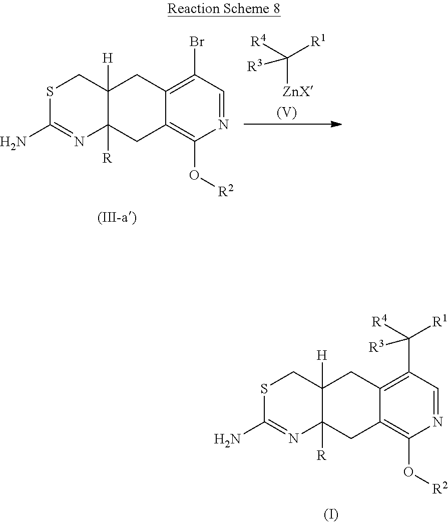

### Pharmacology

The compounds of the present invention and the pharmaceutically acceptable compositions thereof inhibit BACE and therefore may be useful in the treatment or prevention of Alzheimer's Disease (AD), mild cognitive impairment (MCI), senility, dementia, dementia with Lewy bodies, cerebral amyloid angiopathy, multi-infarct dementia, Down's syndrome, dementia associated with Parkinson's disease, dementia of the Alzheimer's type, vascular dementia, dementia due to HIV disease, dementia due to head trauma, dementia due to Huntington's disease, dementia due to Pick's disease, dementia due to Creutzfeldt-Jakob disease, frontotemporal dementia, dementia pugilistica, dementia associated with beta-amyloid and age related macular degeneration, type 2 diabetes and other metabolic disorders.

As used herein, the term “treatment” is intended to refer to all processes, wherein there may be a slowing, interrupting, arresting or stopping of the progression of a disease or an alleviation of symptoms, but does not necessarily indicate a total elimination of all symptoms.

The invention also relates to a compound according to the general Formula (I) or (II), a stereoisomeric form thereof or a pharmaceutically acceptable acid or base addition salt thereof, for use in the treatment or prevention of diseases or conditions selected from the group consisting of AD, MCI, senility, dementia, dementia with Lewy bodies, cerebral amyloid angiopathy, multi-infarct dementia, Down's syndrome, dementia associated with Parkinson's disease, dementia of the Alzheimer's type, dementia associated with beta-amyloid and age related macular degeneration, type 2 diabetes and other metabolic disorders.

The invention also relates to a compound according to the general Formula (I) or (II), a stereoisomeric form thereof or a pharmaceutically acceptable acid or base addition salt thereof, for use in the treatment, prevention, amelioration, control or reduction of the risk of diseases or conditions selected from the group consisting of AD, MCI, senility, dementia, dementia with Lewy bodies, cerebral amyloid angiopathy, multi-infarct dementia, Down's syndrome, dementia associated with Parkinson's disease, dementia of the Alzheimer's type, dementia associated with beta-amyloid and age related macular degeneration, type 2 diabetes and other metabolic disorders.

As already mentioned hereinabove, the term “treatment” does not necessarily indicate a total elimination of all symptoms, but may also refer to symptomatic treatment in any of the disorders mentioned above. In view of the utility of the compound of Formula (I) or (II), there is provided a method of treating subjects such as warm-blooded animals, including humans, suffering from or a method of preventing subjects such as warm-blooded animals, including humans, suffering from any one of the diseases mentioned hereinbefore.

Said methods comprise the administration, i.e. the systemic or topical administration, preferably oral administration, of a therapeutically effective amount of a compound of Formula (I) or (II), a stereoisomeric form thereof, a pharmaceutically acceptable addition salt or solvate thereof, to a subject such as a warm-blooded animal, including a human.

Therefore, the invention also relates to a method for the prevention and/or treatment of any of the diseases mentioned hereinbefore comprising administering a therapeutically effective amount of a compound according to the invention to a subject in need thereof.

The invention also relates to a method for modulating beta-site amyloid cleaving enzyme activity, comprising administering to a subject in need thereof, a therapeutically effective amount of a compound according to the invention and as defined in the claims or a pharmaceutical composition according to the invention and as defined in the claims.

A method of treatment may also include administering the active ingredient on a regimen of between one and four intakes per day. In these methods of treatment the compounds according to the invention are preferably formulated prior to administration. As described herein below, suitable pharmaceutical formulations are prepared by known procedures using well known and readily available ingredients.

The compounds of the present invention, that can be suitable to treat or prevent Alzheimer's disease or the symptoms thereof, may be administered alone or in combination with one or more additional therapeutic agents. Combination therapy includes administration of a single pharmaceutical dosage formulation which contains a compound of Formula (I) or (II) and one or more additional therapeutic agents, as well as administration of the compound of Formula (I) or (II) and each additional therapeutic agent in its own separate pharmaceutical dosage formulation. For example, a compound of Formula (I) or (II) and a therapeutic agent may be administered to the patient together in a single oral dosage composition such as a tablet or capsule, or each agent may be administered in separate oral dosage formulations.

A skilled person will be familiar with alternative nomenclatures, nosologies, and classification systems for the diseases or conditions referred to herein. For example, the fifth edition of the Diagnostic & Statistical Manual of Mental Disorders (DSM-5′) of the American Psychiatric Association utilizes terms such as neurocognitive disorders (NCDs) (both major and mild), in particular, neurocognitive disorders due to Alzheimer's disease, due to traumatic brain injury (TBI), due to Lewy body disease, due to Parkinson's disease or to vascular NCD (such as vascular NCD present with multiple infarctions). Such terms may be used as an alternative nomenclature for some of the diseases or conditions referred to herein by the skilled person.

### Pharmaceutical Compositions

The present invention also provides compositions for preventing or treating diseases in which inhibition of beta-secretase is beneficial, such as Alzheimer's disease (AD), mild cognitive impairment, senility, dementia, dementia with Lewy bodies, Down's syndrome, dementia associated with stroke, dementia associated with Parkinson's disease and dementia associated with beta-amyloid and age related macular degeneration, type 2 diabetes and other metabolic disorders. Said compositions comprising a therapeutically effective amount of a compound according to formula (I) or (II) and a pharmaceutically acceptable carrier or diluent.

While it is possible for the active ingredient to be administered alone, it is preferable to present it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions of this invention may be prepared by any methods well known in the art of pharmacy. A therapeutically effective amount of the particular compound, in base form or addition salt form, as the active ingredient is combined in intimate admixture with a pharmaceutically acceptable carrier, which may take a wide variety of forms depending on the form of preparation desired for administration. These pharmaceutical compositions are desirably in unitary dosage form suitable, preferably, for systemic administration such as oral, percutaneous or parenteral administration; or topical administration such as via inhalation, a nose spray, eye drops or via a cream, gel, shampoo or the like. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. In the compositions suitable for percutaneous administration, the carrier optionally comprises a penetration enhancing agent and/or a suitable wettable agent, optionally combined with suitable additives of any nature in minor proportions, which additives do not cause any significant deleterious effects on the skin. Said additives may facilitate the administration to the skin and/or may be helpful for preparing the desired compositions. These compositions may be administered in various ways, e.g., as a transdermal patch, as a spot-on or as an ointment.

It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

The exact dosage and frequency of administration depends on the particular compound of formula (I) or (II) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

The present compounds can be used for systemic administration such as oral, percutaneous or parenteral administration; or topical administration such as via inhalation, a nose spray, eye drops or via a cream, gel, shampoo or the like. The compounds are preferably orally administered. The exact dosage and frequency of administration depends on the particular compound according to formula (I) or (II) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

The amount of a compound of Formula (I) or (II) that can be combined with a carrier material to produce a single dosage form will vary depending upon the disease treated, the mammalian species, and the particular mode of administration. However, as a general guide, suitable unit doses for the compounds of the present invention can, for example, preferably contain between 0.1 mg to about 1000 mg of the active compound. A preferred unit dose is between 1 mg to about 500 mg. A more preferred unit dose is between 1 mg to about 300 mg. Even more preferred unit dose is between 1 mg to about 100 mg. Such unit doses can be administered more than once a day, for example, 2, 3, 4, 5 or 6 times a day, but preferably 1 or 2 times per day, so that the total dosage for a 70 kg adult is in the range of 0.001 to about 15 mg per kg weight of subject per administration. A preferred dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

For the compositions, methods and kits provided above, one of skill in the art will understand that preferred compounds for use in each are those compounds that are noted as preferred above. Still further preferred compounds for the compositions, methods and kits are those compounds provided in the non-limiting Examples below.

### Experimental Part

Hereinafter, the term“aq.” means aqueous, “r.m.” means reaction mixture, “r.t.” or “RT” mean room temperature, “DIPEA” means N,N-diisopropylethylamine, “DIPE” means diisopropylether, “THF” means tetrahydrofuran, “DMF” means dimethylformamide, “DCM” means dichloromethane, “EtOH” means ethanol “EtOAc” means ethylacetate, “AcOH” means acetic acid, “iPrOH” means isopropanol, “iPrNH2” means isopropylamine, “MeCN” means acetonitrile, “MeOH” means methanol, “Pd(OAc)₂” means palladium(II)diacetate, “rac” means racemic, “sat.” means saturated, “SFC” means supercritical fluid chromatography, “SFC-MS” means supercritical fluid chromatography/mass spectrometry, “LC-MS” means liquid chromatography/mass spectrometry, “GCMS” means gas chromatography/mass spectrometry, “HPLC” means high-performance liquid chromatography, “RP” means reversed phase, “UPLC” means ultra-performance liquid chromatography, “Rₜ” means retention time (in minutes), “[M+H]⁺” means the protonated mass of the free base of the compound, “DAST” means diethylaminosulfur trifluoride, “DMTMM” means 4-(4,6-dimethoxy-1,3,5-triazin-2-yl)-4-methylmorpholinium chloride, “HATU” means O-(7-azabenzotriazol-1-yl)-N,N,N′,N′-tetramethyluronium hexafluorophosphate, “HBTU” means N,N,N,N′-Tetramethyl-O-(1H-benzotriazol-1-yl)uronium hexafluorophosphate, “Xantphos” means (9,9-dimethyl-9H-xanthene-4,5-diyl)bis[diphenylphosphine], “TFA” means trifluoroacetic acid, “Et₂O” means diethylether, “DMSO” means dimethylsulfoxide, “NMR” means nuclear magnetic resonance, “LDA” means lithium diisopropylamide, “DIPA” means diisopropylamine, “n-BuLi” means n-butyllithium. “h” means hours. “min” means minutes, “sol.” means solution, “BOC” means t-butoxycarbonyl, “DMAP” means dimethylaminopyridine, “NBS” means N-bromosuccinimide, “Pd(PPh₃)₄” means tetrakis(triphenylphosphine)palladium(0), “RuPhos” means [[2-Dicyclohexylphosphino-2′,6′-diisopropoxybiphenyl]], “DBU” means 1,8-diazabicyclo[5.4.0]undec-7-ene, “SQD” means Single Quadrupole Detector, “MSD” means Mass Selective Detector, “BEH” means bridged ethylsiloxane/silica hybrid, “DAD” means Diode Array Detector, “HSS” means High Strength silica, “Q-Tof” means Quadrupole Time-of-flight mass spectrometers,

“CLND” means ChemiLuminescent Nitrogen Detector, “ELSD” means Evaporative Light Scanning Detector.

For the synthesis of intermediates 13 and 14 and compound 10, flow chemistry reactions were performed in a Vapourtec R2+R4 unit using standard reactors provided by the vendor.

### Assignment and Graphical Representation of Stereochemical Configuration

The stereoconfiguration of centres C₄ₐ and C₁₀ₐ of intermediates/compounds has been represented as follows:

a) when the intermediate/compound is enantiopure and the absolute stereoconfiguration is known, the core has been represented as

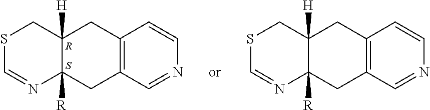

when for instance, the stereoconfiguration corresponds with C₄ₐ(R),C₁₀ₐ(S) and the compound is a single diastereoisomer and enantiopure;
b) when the intermediate/compound is enantiopure but the absolute stereoconfiguration has not been determined, the core has been represented as

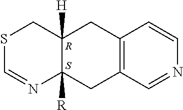

(wherein the wedges have been assigned at random to indicate the cis diastereoisomer); when the other pure enantiomer of cis relative configuration has been isolated, the intermediate/compound has been represented as

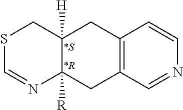

in order to differentiate from the other isolate enantiopure intermediate/compound;
c) when the intermediate/compound is a racemic mixture of two enantiomers of cis relative configuration, the core has been represented as

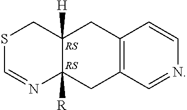

The absolute stereochemical configuration of intermediates/compounds has been rationalized on the basis of chemical synthetic methods and NMR (assignment of relative stereoconfiguration) and co-crystallisation of various enantiopure analogues with BACE 1 enzymes, which enabled ascertaining the preferred orientation of the R group in the compounds, together with the exhibited in vitro activity of the compounds.

### A. Preparation of the Intermediates

### Intermediate 1

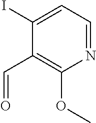

A mixture of DIPA (3.5 mL, 25 mmol) in THF (100 mL) was cooled to −20° C. and n-BuLi (2.7 M in heptane, 9.2 mL, 25 mmol) was added dropwise. After stirring 10 min, the r.m. was cooled to −75° C. and 2-fluoro-3-iodopyridine (5.55 g, 25 mmol) in THF (50 mL) was added dropwise. Stirring was continued for 2 h at −65° C. The r.m. was cooled to −75° C. and ethyl formate (2.3 mL, 28 mmol) in THF (25 mL) was added dropwise. After 10 min sodium methoxide (5.8 mL, 0.95 g/mL, 25 mmol, 25% purity) was added dropwise. The cooling bath was removed and the r.m. was allowed to come to r.t. and treated with brine (50 mL), Et₂O (100 mL) and the layers were separated. The aq. layer was extracted with Et₂O (100 mL) and the combined organic layers were treated with brine (50 mL), dried over MgSO₄, filtered and concentrated in vacuo to afford intermediate 1 (6.15 g, 94%), which was used as such in the next reaction step.

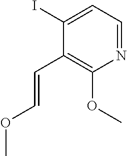

### Intermediate 2

To a stirred mixture of methoxymethyl triphenylphosphonium chloride (8.4 g, 24 mmol) in toluene (150 mL) was added potassium bis(trimethylsilyl)amide (0.7 M in toluene, 34 mL, 24 mmol) dropwise at −10° C. Stirring was continued for 30 min at this temperature. Intermediate 1 (2.1 g, 8 mmol) in toluene (20 mL) was added dropwise and after 2 h the r.m. was quenched with water (50 mL) and the layers were separated. The organic layer was dried over MgSO₄, filtered and concentrated in vacuo to afford a tan oil. This oil was purified by column chromatography (silica, EtOAc/heptane 0/100 to 10/90) to afford intermediate 2 as an oil (1.86 g, 80%).

### Intermediate 3

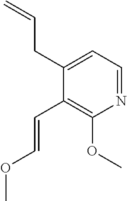

To a stirred and cooled (−70° C.) mixture of intermediate 2 (30 g, 100 mmol) in THF (500 mL) was added dropwise isopropylmagnesium chloride-lithium chloride complex (105 mL, 1.3 M, 140 mmol) while keeping the internal temperature below −65° C. When addition was complete, stirring was continued for 1.5 h. Copper(I) cyanide di(lithium chloride) complex sol. (105 mL, 1 M, 110 mmol) was then added dropwise at −70° C. and after 15 min allyl bromide (28 mL, 31 mmol) was added dropwise. The r.m. was allowed to come to r.t. and then quenched with brine (100 mL), diluted with Et₂O (0.3 L) and water (0.1 L) and the layers were separated. The organic layer was washed first portionwise with ammonia until the blue colour disappeared (5×0.2 L) and then with brine (0.1 L). The organic layer was dried over MgSO₄, filtered and concentrated in vacuo to afford a residue which was purified by column chromatography (silica, DCM/heptane 98/2 to 100/0) to afford intermediate 3 (19.6 g, 93%).

### Intermediate 4

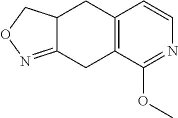

A stirred sol. of intermediate 3 (19.6 g, 95 mmol) in THF (200 mL) was treated with aq. 6 M HCl (70 mL, 420 mmol) and the r.m. was heated at 50° C. for 30 min. The r.m was poured into ice water (0.2 L) and treated with sat. Na₂CO₃ sol. until neutral pH. The r.m. was extracted with DCM (3×0.1 L) and the combined organic layers were dried over MgSO₄. To the resulting sol. was added triethylamine (40 mL, 290 mmol) and then hydroxylamine hydrochloride (8 g, 120 mmol) and stirring was continued for 1 h. The r.m. was diluted with sat. NaHCO₃sol. (0.1 L) and the layers were separated. The organic layer was dried over MgSO₄, filtered and transferred to a 1 L 4 neck flask, equipped with a mechanical stirrer and cooled to 0° C. (internal temperature). To this cooled sol., sodium hypochlorite (210 mL, 470 mmol) was added dropwise. After complete addition, the r.m. was allowed to come to r.t. and stirring was continued at r.t. overnight. The layers were separated and the aq. layer was extracted with DCM (0.2 L). The combined organic layers were dried over MgSO₄, filtered and concentrated in vacuo to give a solid which was recrystallized from DIPE (0.1 L) to afford intermediate 4 (8.64 g, 44%).

### Intermediate 5

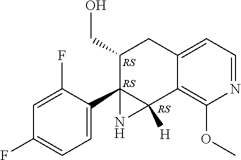 [SMILES: COc1nccc2c1[C@@H]1N[C@]1(c1ccc(F)cc1F)[C@H](CO)C2]

1-Bromo-2,4-difluorobenzene (9.7 mL, 70.5 mmol) was stirred in THF (43 mL) under nitrogen atmosphere and cooled to −15° C. Isopropylmagnesium chloride (2 M in THF, 43 mL, 86.1 mmol) was added dropwise at −15° C. The r.m. was further stirred at 0-5° C. for 1 h, then cooled again to −15° C. and intermediate 4 (7.2 g, 35.26 mmol) dissolved in THF (43 mL) was added dropwise. The mixture was then allowed to reach r.t., added dropwise to 60 ml of NH₄Cl sat. sol. and extracted with EtOAc. The organic layer was dried over MgSO₄, filtered and concentrated in vacuo to afford intermediate 5 (11.15 g, 99%).

### Intermediate 6

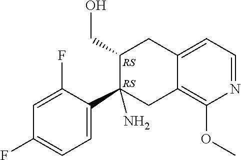 [SMILES: COc1nccc2c1C[C@@](N)(c1ccc(F)cc1F)[C@H](CO)C2]

Raney®-Nickel (64 g) and thiophene (4% in DIPE, 85 mL) in EtOH (473 mL) were placed in a hydrogenation flask before intermediate 5 (17.2 g, 54 mmol) dissolved in EtOH(473 mL) was added. The flask was degassed and then flushed with hydrogen gas before being stirred for 6 h at 14° C. The r.m. was filtered over Dicalite® and washed with EtOH and THF before the product was concentrated by evaporation. The product was purified (silica, MeOH/DCM 0/100 to 6/94). The pure fractions were evaporated to yield intermediate 6 (10.34 g, 60%).

### Intermediate 7

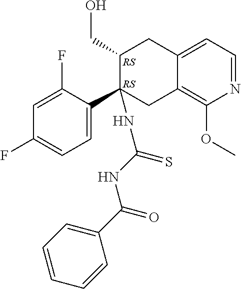 [SMILES: COc1nccc2c1C[C@@](NC(=S)NC(=O)c1ccccc1)(c1ccc(F)cc1F)[C@H](CO)C2]

Intermediate 6 (10.34 g, 32 mmol) was dissolved in DCM (130 mL) in an ice bath before benzoyl isothiocyanate (7.38 g, 45.19 mmol) in DCM (20 mL) was added dropwise to the mixture and the r.m. was allowed to stir at r.t. for 1.5 h. A small amount of ice was added to the still stirring r.m. and the product was extracted using DCM; the organic layer was dried over MgSO₄, filtered and concentrated by evaporation. The organic layer was purified by column chromatography (silica, EtOAc/heptane 0/100 to 80/20). The fractions containing product were collected and concentrated by evaporation to yield intermediate 7 (15 g, 96%).

### Intermediates 8 and 8A

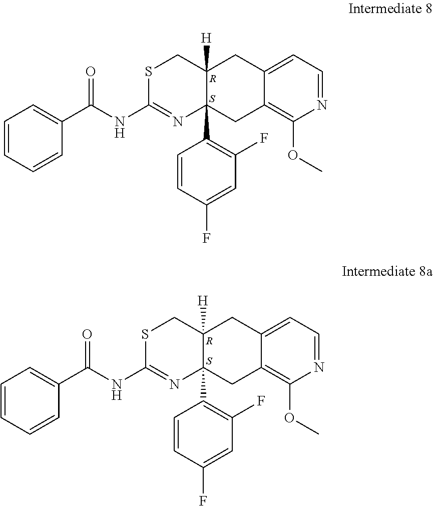

Intermediate 7 (3.5 g, 7.24 mmol) was stirred in DCM (91 mL) at r.t. under a flow of nitrogen before 1-chloro-N,N,2-trimethylpropenylamine (2.62 mL, 19.80 mmol) was added dropwise and the r.m. was allowed to stir for 10 min. The reaction went to completion and was then quenched with 20 mL of sat. aq. sol. NaHCO₃ and allowed to stir for 10 min. The organic material was extracted using DCM, dried over MgSO₄, filtered and concentrated by evaporation. This material was stirred in DIPE to afford a white solid which was filtered off and dried in the oven to yield 2.46 g of a mixture which was purified by Prep SFC (Stationary phase: Chiralpak Diacel AD 30×250 mm, mobile phase: CO₂, MeOH with 0.2% iPrNH₂) to yield intermediate 8 (1.99 g, 33%, pure enantiomer) and intermediate 8a (1.67, 28% pure enantiomer).

### Intermediate 9

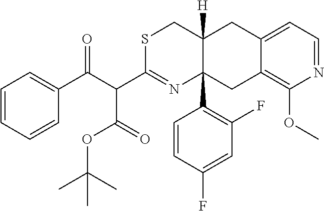 [SMILES: COc1nccc2c1C[C@]1(c3ccc(F)cc3F)N=C(C(C(=O)OC(C)(C)C)C(=O)c3ccccc3)SC[C@@H]1C2]

A stirred mixture of intermediate 8 (2.2 g, 0.0047 mol) in THF (20 mL, 0.89 g/mL, 0.25 mol) was treated with BOC-anhydride (1.24 g, 0.0057 mol) and DMAP (50 mg, 0.00041 mol). After stirring for 1 h at r.t., the r.m. was diluted with saturated NaHCO₃ solution (20 mL), water (50 mL) and EtOAc (100 mL) and the layers were separated. The aqueous layer was extracted with EtOAc (50 mL). The combined organic layers were treated with brine (20 mL), dried over MgSO₄, filtered and concentrated in vacuo to give intermediate 9 as a white foam (2.77 g, 99%).

### INTERMEDIATE 10

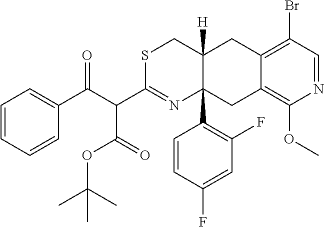 [SMILES: COc1ncc(Br)c2c1C[C@]1(c3ccc(F)cc3F)N=C(C(C(=O)OC(C)(C)C)C(=O)c3ccccc3)SC[C@@H]1C2]

To a stirred mixture of intermediate 9 (2.77 g, 0.0049 mol) in ACN (250 mL, 0.79 g/mL, 4.81 mol) was added N-bromosuccinimide (1 g, 0.0056 mol) in small portions and the ensuing r.m. was stirred for 4 days at r.t. then more N-bromosuccinimide (0.2 g, 0.0011 mol) was added and stirring was continued for another 3 h. The r.m. was diluted with 40 mL of saturated NaHCO₃, water (0.1 L), EtOAc (200 mL) and the layers were separated. The aqueous layer was extracted with EtOAc (50 mL) and the combined organic layers were treated with brine (0.1 L), dried over MgSO₄, filtered and concentrated in vacuo to afford an off white solid. This was purified by silica gel column chromatography using a 120 g Redisep flash column eluting with a gradient of 0-50% EtOAc in heptane to afford intermediate 10 as a bright white solid (2.1 g, yield 67%).

### Intermediate 11

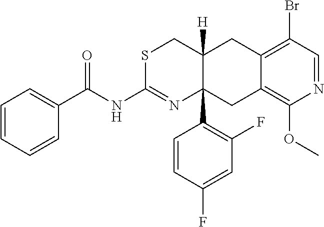 [SMILES: COc1ncc(Br)c2c1C[C@]1(c3ccc(F)cc3F)N=C(NC(=O)c3ccccc3)SC[C@@H]1C2]

Intermediate 10 (13.71 g, 21 mmol) and formic acid (79.7 mL, 2.1 mmol) were stirred at r.t. for 1 h. The formic acid present in the r.m. was evaporated and the product was basified with Na₂CO₃ before being extracted with DCM. The organic layer was dried over MgSO₄, filtered and concentrated by evaporation to yield a product that was crystallized from DIPE. The crystals were filtered off and dried, yielding intermediate 11 (9.32 g, 81%).

### Intermediate 12

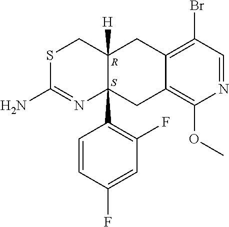

Intermediate 11 (9.32 g, 17 mmol), DBU (25.5 mL, 171 mmol) and MeOH (192.8 mL) were placed in a pressure tube and stirred at 60° C. overnight. The r.m. was concentrated by evaporation before the material was purified twice by column chromatography (silica, DCM to 5% MeOH in DCM). The fractions containing product were combined and concentrated by evaporation to yield intermediate 12 (6.84 g, 91%).

### Intermediate 13

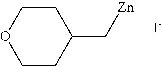

A solution of 4-(iodomethyl)tetrahydro-2H-pyran ([101691-94-5], 565 mg, 3 mmol) in LiCl solution (0.5 M in THF, 5 mL, 3 mmol) was pumped using the R2+R4 through a column containing activated Zn (12 g) at 0.5 mL/min at 60° C. Titration with I₂/LiCl showed a concentration of organozinc reagent of 0.28 M. The solution was used in the next step without further purification.

### Intermediate 14

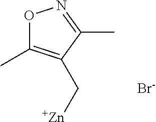

A solution of 4-(bromomethyl)-3,5-dimethylisoxazole (475.1 mg, 3 mmol) in THF (5 mL) was pumped using the R2+R4 through a column containing activated Zn at 0.5 mL/min at 40° C. The solution was used as such in the next step.

### B. Preparation of the Compounds

### EXAMPLE 1—PREPARATION OF COMPOUND 1

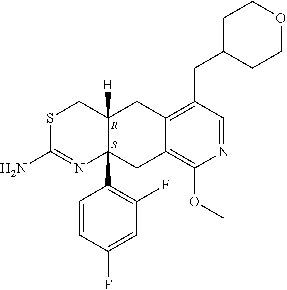

A solution of intermediate 13 (608 μL, 0.28 M, 0.2 mmol) was added to a solution of intermediate 12 (25 mg, 0.06 mmol), Pd(OAc)₂ (0.64 mg, 0.003 mmol) and RuPhos (2.65 mg, 0.006 mmol) in THF (0.25 mL). The mixture was heated at 100° C. for 10 min under microwave irradiation. The mixture was quenched with 10% NH₄Cl and extracted with EtOAc. The organic layer was separated, dried over Na₂SO₄, filtered and the solvent evaporated. The residue was purified by column chromatography (silica, EtOAc/DCM 0/100 to 100/0). Desired fractions were collected and the solvent evaporated to yield a product that was further purified by RP HPLC (stationary phase: C18 Sunfire 30×100 mm 5 μm, mobile phase: NH₄HCO₃/ACN), yielding compound 1 (2 mg, 8%) as an off white foam.

### EXAMPLE 2—PREPARATION OF COMPOUND 2

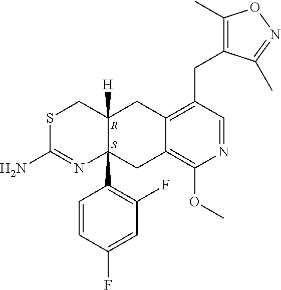

A solution of intermediate 14 (0.27 M, 673 μL, 0.2 mmol) was added to a solution of intermediate 12 (20 mg, 0.05 mmol), Pd(OAc)₂ (1.02 mg, 0.005 mmol) and RuPhos (4.2 mg, 0.009 mmol) in THF (0.25 mL). The mixture was heated in a microwave oven for 10 min at 100° C. The mixture was quenched with 10% NH₄Cl and extracted with EtOAc. The organic layer was separated, dried over Na₂SO₄, filtered and the solvent evaporated. The residue was purified by column chromatography (silica, EtOAc/DCM 0/100 to 100/0). Desired fractions were collected and the solvent evaporated to yield a product that was further purified by RP HPLC (stationary phase: C18 Sunfire 30×100 mm 5 μm, mobile phase: NH₄HCO₃/ACN) to yield compound 2 (3.4 mg (16%) as an off white foam.

### EXAMPLE 3—PREPARATION OF COMPOUND 5

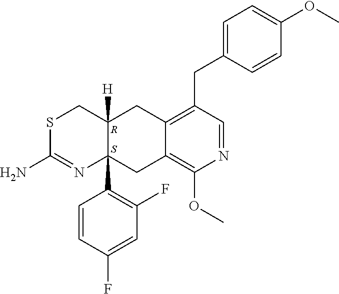

Procedure a: Intermediate 12 (50 mg) was dissolved in (4-methoxybenzyl)zinc(II) chloride solution (0.5 M, 1.1 mL). RuPhosPdG2 (5 mg) was added and the reaction was heated at 80° C. for 30 min under microwave irradiation. The mixture was poured into water and extracted with DCM. The organics were derived, filtered an evaporated to give a gum which was purified by RP HPLC (stationary phase: C18(2) Phenomenex Luna® 150 mm×21.2 mm 5 μm; mobile phase 10 mM CH₃CO₂NH₄ solution in water) to give compound 5 (9 mg, 16%) as a white solid.

### EXAMPLE 4—PREPARATION OF COMPOUND 9

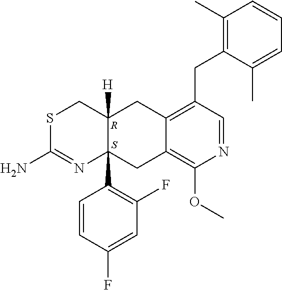

A solution of 2,6-dimethylbenzyl bromide (33.91 mg, 017 mmol) in THF (0.35 mL) was pumped using the R2+R4 through a column containing activated Zinc at 0.5 ml/min at 40° C. The outcome was collected into a solution of intermediate 12 (25 mg, 0.057 mmol), Palladium(II) acetate (0.64 mg, 0.003 mmol), RuPhos (2.65 mg, 0.006 mmol) in THF (0.25 mL) and heated in a microwave oven for 10 min at 100° C. The mixture was quenched with 10% NH₄Cl and extracted with EtOAc. The organic layer was separated, dried (Na₂SO₄), filtered and the solvent evaporated. The residue was purified by column chromatography (silica, EtOAc/DCM 0/100 to 100/0). Desired fractions were collected and the solvent evaporated to yield crude compound 9, which was further purified by RP HPLC (Stationary phase: C18 Sunfire 30×100 mm 5 μm, mobile phase: NH₄HCO₃/CH₃CN) to yield compound 9 (9 mg, 33%) as an off white foam.

### EXAMPLE 5—PREPARATION OF COMPOUND 10

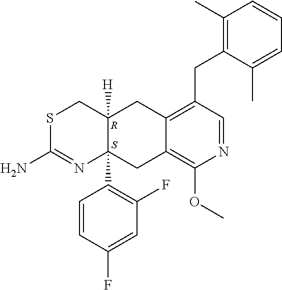

The synthesis of compound 10 was performed in an analogous manner to the synthesis of compound 5, starting from intermediate 9 and subjecting it to the same synthetic transformations as intermediates 8 to 12, and then reacting the pure enantiomer of intermediate 12 to a Negishi coupling with 2,6-dimethylbenzylzinc(II) bromide generated in situ (by passing a solution of 2,6-dimethylbenzyl bromide in THF using the R2+R4 through a column containing activated Zn at 0.5 ml/min at 40° C.) to yield compound 10 (40 mg, 73%).

### EXAMPLE 6

### Preparation of Compounds According to General Procedure Flow Chemistry

Intermediate 12 (20 mg) was placed in one vessel in solvent (e.g. NMP (400 μL)). In a second vessel the bromo or chloro Negishi zincate (e.g. benzylzinc(II) bromide (400 μL, 0.5M in THF)) was added and in a third vessel the catalyst in solvent (e.g. Pd(dppf)Cl₂ (1.7 mg, 0.05 eq.) in THF (400 μL)) was prepared. The three vessels were loaded onto a Gilson 215 and injected into 250 μl injection loops and subsequently onto a 2 ml stainless steel coil heated to 80° C. with each pump running at 33 μL/min. The outflow injected automatically through a 20 μL loop into the purification and assay part of the platform.

Tables 1 and 2 below list the compounds of Formula (I) and (II) that were exemplified (*Ex. No.) and prepared by analogy to one of the above Examples (indicated by the Ex. No.). In case no salt form is indicated, the compound was obtained as a free base. ‘Ex. No.’ refers to the Example number according to which protocol the compound was synthesized. ‘Co. No.’ means compound number.

TABLE 1
Compounds of Formula (I) of C₄ₐ(R), C₁₀ₐ(S) configuration

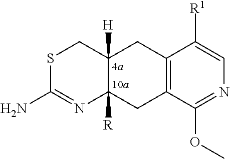

| Co. | Ex. | | |
|---|---|---|---|
| No. | No. | R¹ | R |
| 1 | *E1 | 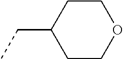 | 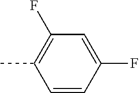 |
| 2 | *E2 | 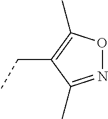 | 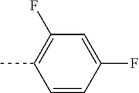 |
| 3 | E6 | 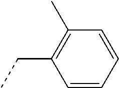 | 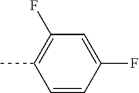 |
| 4 | E6 | 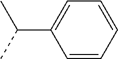 | 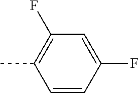 |
| 5 | *E3 | 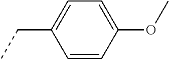 | 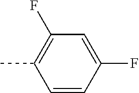 |
| 6 | E6 | 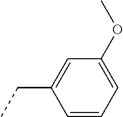 | 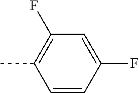 |
| 7 | E6 | 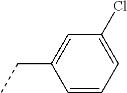 | 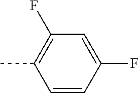 |
| 8 | E6 | 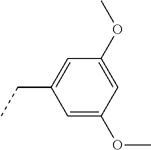 | 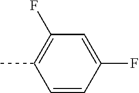 |
| 9 | *E4 | 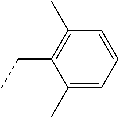 | 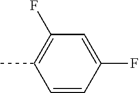 |

TABLE 2
Compounds of Formula (I) of C₄ₐ(S), C₁₀ₐ(R) configuration

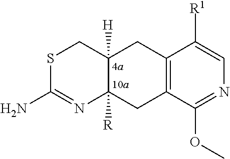

| Co. | Ex. | | |
|---|---|---|---|
| No. | No. | R¹ | R |
| 10 | *E5 | 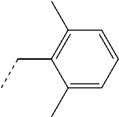 | 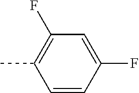 |

### C. Analytical Part

### Melting Points

Values are either peak values or melt ranges, and are obtained with experimental uncertainties that are commonly associated with this analytical method.

For a number of compounds, melting points were determined with a DSC823e (Mettler-Toledo). Melting points were measured with a temperature gradient of 10° C./minute. Maximum temperature was 300° C.

### LCMS

### LCMS General Procedure 1

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

Compounds are described by their experimental retention times (Rₜ) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, etc. . . . ). For molecules with multiple isotopic patterns (Br, Cl.), the reported value is the one obtained for the lowest isotope mass. All results were obtained with experimental uncertainties that are commonly associated with the method used.

Hereinafter, “SQD” means Single Quadrupole Detector, “MSD” Mass Selective Detector, “RT” room temperature, “BEH” bridged ethylsiloxane/silica hybrid, “DAD” Diode Array Detector, “HSS” High Strength silica., “Q-Tof” Quadrupole Time-of-flight mass spectrometers, “CLND”, ChemiLuminescent Nitrogen Detector, “ELSD” Evaporative Light Scanning Detector,

TABLE 3
LCMS Method codes, general procedure 1 (Flow expressed in mL/min; column temperature (T) in ° C.; Run time in minutes)

| | | | | | FLOW | RUN TIME |
|---|---|---|---|---|---|---|
| METHOD | INSTRUMENT | COLUMN | MOBILE PHASE | GRADIENT | COLT | (MIN) |
| 1 | Waters: Acquity ® | Waters: CSH ™ C18 | A: 95% CH₃COONH₄ | From 95% A to 5% A | 1 | 5 |
| | IClass UPLC ® - DAD | (1.7 μm, 2.1 × 50 mm) | 6.5 mM + 5% CH₃CN, | in 4.6 min, held for | −50 | |
| | and Xevo G2-S QTOF | | B: CH₃CN | 0.4 min | | |
| 2 | Waters: Acquity ® | Waters: BEH C18 | A: 10 mM CH₃COONH₄ 95% | From 95% A to 5% A | 0.7 | 1.8 |
| | UPLC ® - DAD and | (1.7 μm, 2.1*50 mm) | H₂O + 5% CH₃CN | in 1.3 min, held for | −70 | |
| | SQD | | B: CH₃CN | 0.2 min, to 95% A in | | |
| | | | | 0.2 min held for | | |
| | | | | 0.1 min | | |

TABLE 4
Analytical data, general procedure 1 - Rt means retention
time (in minutes), [M + H]⁺ means the protonated
mass of the compound, method refers to the method used for (LC)MS.

| Co. | Rt | | | |
|---|---|---|---|---|
| No. | (min) | [M + H]⁺ | [M − H]⁻ | Method |
| 1 | 2.16 | 460.2 | 458.2 | 1 |
| 2 | 2.13 | 471.2 | 469.2 | 1 |
| 9 | 3 | 480.2 | 478.2 | 1 |
| 10 | 3.03 | | 478.2 | 1 |
| 5 | 1.3175 | 482.3 | 480.4 | 2 |

### LCMS General Procedure 2

HPLC-MS was carried out using an Acquity™ Ultra Performance LC system, comprising a PDA detector, Binary Solvent Manager and SQ detector (Waters UK Ltd., Elstree, UK), tandem linked to a mass spectrometry system (Waters UK Ltd., Manchester, UK) employing vendor software (OpenLynx Browser™ v4.1, SQ Detector v4.1, Instrument Driver V4.1 and MassLynx™ v4.1). Parallel evaporative light-scattering detection (385-LC, Varian; Agilent Technologies, Wokingham, U.K.) was incorporated into the system via an active splitter (Model EHMA, 10-port valve; Valco Intruments, active split achieved by proprietary Cyclofluidic hardware). Direct injection mass spectrometry was carried out on a ThermoQuest Finnigan LCQduo employing Xcalibur® v2.0 SR2, Tune Plus v2.0 and Qual Broswer v2.0 vendor software (ThermoFisher).

TABLE 3a
Conditions adopted, general procedure 2:

| | |
|---|---|
| Column | Phenomenex Luna C18(2) 5 μm 150 × 4.6 mm. |
| Eluent | Aqueous phase - Water containing 0.2% v/v trifluoroacetic |
| | acid. |
| | Organic phase - Acetonitrile containing 0.2% v/v |
| | trifluoroacetic acid. |
| Temperature | Ambient |
| Detection | Mass spectrometry - ESI + over m/z range 150 to 850. |
| | UV - Diode array over range 220 to 400 nm. |
| | ELSD - Evaporator at 35° C., nebuliser at 35° C. |
| | and gas flow at 1.8 L/min. |

Equilibration was achieved using a start-up method ahead of the next sample run.

TABLE 4a
Analytical data, general procedure 2 - Rₜ means
retention time (in minutes), [M + H]⁺
means the protonated mass of the compound.

| Co. No. | Rt (min) | [M + H]⁺ | UV area % |
|---|---|---|---|
| 3 | 7.02 | 466.2 | 100 |
| 4 | | 466.2 | 100 |
| 5 | | 482.2 | 98 |
| 6 | 6.63 | 482.2 | 82 |
| 7 | 7.16 | 486.1 | 100 |
| 8 | 6.64 | 512.2 | 100 |

### NMR

For a number of compounds, ¹H NMR spectra were recorded on a Bruker Avance III with a 300 MHz Ultrashield magnet, on a Bruker DPX-400 spectrometer operating at 400 MHz, on a Bruker Avance I operating at 500 MHz, on a Bruker DPX-360 operating at 360 MHz, or on a Bruker Avance 600 spectrometer operating at 600 MHz, using CHLOROFORM-d (deuterated chloroform, CDCl₃) or DMSO-d₆ (deuterated DMSO, dimethyl-d6 sulfoxide) as solvent. Chemical shifts (6) are reported in parts per million (ppm) relative to tetramethylsilane (TMS), which was used as internal standard.

TABLE 5
¹H NMR results

| Co. No. | ¹H NMR result |
|---|---|
| 2 | ¹H NMR (400 MHz, BENZENE-d₆) δ ppm 2.23 (dd, J = 16.0, |
| | 5.3 Hz, 1 H) 2.45-2.60 (m, 2 H) 3.15-3.23 (m, 1 H) 3.26 (dd, |
| | J = 10.7, 1.9 Hz, 1 H) 3.30-3.39 (m, 1 H) 3.51 (dd, J = 10.7, |
| | 2.7 Hz, 1 H) 3.82 (s, 3 H) 6.34 (d, J = 5.2 Hz, 1 H) 6.46-6.58 |
| | (m, 2 H) 7.49 (td, J = 9.1, 6.7 Hz, 1 H) 7.97 (d, J = 5.2 Hz, 1 H) |

### D. Pharmacological Examples

The compounds provided in the present invention are inhibitors of the beta-site APP-cleaving enzyme 1 (BACE1). Inhibition of BACE1, an aspartic protease, is believed to be relevant for treatment of Alzheimer's Disease (AD). The production and accumulation of beta-amyloid peptides (Abeta) from the beta-amyloid precursor protein (APP) is believed to play a key role in the onset and progression of AD. Abeta is produced from the amyloid precursor protein (APP) by sequential cleavage at the N- and C-termini of the Abeta domain by beta-secretase and gamma-secretase, respectively.

Compounds of Formula (I) and (II) are expected to have their effect substantially at BACE1 by virtue of their ability to inhibit the enzymatic activity. The behaviour of such inhibitors tested using a biochemical Fluorescence Resonance Energy Transfer (FRET) based assay and a cellular aLisa assay in SKNBE2 cells described below and which are suitable for the identification of such compounds, and more particularly the compounds according to Formula (I), are shown in Table 8 and Table 9.

### BACE1 Biochemical FRET Based Assay

This assay is a Fluorescence Resonance Energy Transfer Assay (FRET) based assay. The substrate for this assay is an APP derived 13 amino acids peptide that contains the ‘Swedish’ Lys-Met/Asn-Leu mutation of the amyloid precursor protein (APP) beta-secretase cleavage site. This substrate also contains two fluorophores: (7-methoxycoumarin-4-yl) acetic acid (Mca) is a fluorescent donor with excitation wavelength at 320 nm and emission at 405 nm and 2,4-Dinitrophenyl (Dnp) is a proprietary quencher acceptor. The distance between those two groups has been selected so that upon light excitation, the donor fluorescence energy is significantly quenched by the acceptor, through resonance energy transfer. Upon cleavage by BACE1, the fluorophore Mca is separated from the quenching group Dnp, restoring the full fluorescence yield of the donor. The increase in fluorescence is linearly related to the rate of proteolysis.

Briefly in a 384-well format recombinant BACE1 protein in a final concentration of 0.04 μg/ml is incubated for 450 minutes at room temperature with 20 μM substrate in incubation buffer (50 mM Citrate buffer pH 5.0, 0.05% PEG) in the presence of compound or DMSO. Next the amount of proteolysis is directly measured by fluorescence measurement (excitation at 320 nm and emission at 405 nm) at different incubation times (0, 30, 60, 90, 120 and 450 min). For every experiment a time curve (every 30 min between 0 min and 120 min) is used to determine the time where we find the lowest basal signal of the high control. The signal at this time (Tx) is used to subtract from the signal at 450 min. Results are expressed in RFU, as difference between T450 and Tx.

A best-fit curve is fitted by a minimum sum of squares method to the plot of % Controlmin versus compound concentration. From this an IC₅₀ value (inhibitory concentration causing 50% inhibition of activity) can be obtained.

$$LC = Median of the low control values = Low control \text{:} Reaction without enzyme$$

$$HC = Median of the High control values = High control \text{:} Reaction with enzyme$$

$$\% Effect = 100 \text{-} [ ( sample \text{-} LC ) / ( HC \text{-} LC ) * 100 ]$$

$$\% Control = ( sample / HC ) * 100$$

$$\% Controlmin = ( sample \text{-} LC ) / ( HC \text{-} LC ) * 100$$

The following exemplified compounds were tested essentially as described above and exhibited the following the activity:

TABLE 6

| | Biochemical FRET |
|---|---|
| Co. No. | based assay pIC₅₀ |
| 1 | 8.52 |
| 2 | 8.23 |
| 5 | 7.64 |
| 9 | 7.99 |

### Cellular αLisa Assay in SKNBE2 Cells

In two αLisa assays the levels of Abeta total and Abeta 1-42 produced and secreted into the medium of human neuroblastoma SKNBE2 cells are quantified. The assay is based on the human neuroblastoma SKNBE2 expressing the wild type Amyloid Precursor Protein (hAPP695). The compounds are diluted and added to these cells, incubated for 18 hours and then measurements of Abeta 1-42 and Abeta total are taken. Abeta total and Abeta 1-42 are measured by sandwich αLisa. αLisa is a sandwich assay using biotinylated antibody AbN/25 attached to streptavidin coated beads and antibody Ab4G8 or cAb42/26 conjugated acceptor beads for the detection of Abeta total and Abeta 1-42 respectively. In the presence of Abeta total or Abeta 1-42, the beads come into close proximity. The excitation of the donor beads provokes the release of singlet oxygen molecules that trigger a cascade of energy transfer in the acceptor beads, resulting in light emission. Light emission is measured after 1 hour incubation (excitation at 650 nm and emission at 615 nm).

A best-fit curve is fitted by a minimum sum of squares method to the plot of % Controlmin versus compound concentration. From this an IC₅₀ value (inhibitory concentration causing 50% inhibition of activity) can be obtained.

$$LC = Median of the low control values = Low control \text{:} cells preincubated without compound , without biotinylated Ab in the α Lisa$$

$$HC = Median of the High control values = High control \text{:} cells preincubated without compound$$

$$\% Effect = 100 \text{-} [ ( sample \text{-} LC ) / ( HC \text{-} LC ) * 100 ]$$

$$\% Control = ( sample / HC ) * 100$$

$$\% Controlmin = ( sample \text{-} LC ) / ( HC \text{-} LC ) * 100$$

The following exemplified compounds were tested essentially as described above and exhibited the following the activity:

TABLE 7

| | Cellular αLisa assay in | Cellular αLisa assay in |
|---|---|---|
| | SKNBE2 cells Abeta 42 | SKNBE2 cells Abeta |
| Co. No. | pIC₅₀ | total pIC₅₀ |
| 1 | 9.12 | |
| 2 | 8.58 | |
| 5 | 7.17 | |
| 9 | 7.37 | |

n.t. means not tested

### BACE2 Biochemical FRET Based Assay

This assay is a Fluorescence Resonance Energy Transfer Assay (FRET) based assay. The substrate for this assay contains the ‘Swedish’ Lys-Met/Asn-Leu mutation of the amyloid precursor protein (APP) beta-secretase cleavage site. This substrate also contains two fluorophores: (7-methoxycoumarin-4-yl) acetic acid (Mca) is a fluorescent donor with excitation wavelength at 320 nm and emission at 405 nm and 2,4-Dinitrophenyl (Dnp) is a proprietary quencher acceptor. The distance between those two groups has been selected so that upon light excitation, the donor fluorescence energy is significantly quenched by the acceptor, through resonance energy transfer. Upon cleavage by the beta-secretase, the fluorophore Mca is separated from the quenching group Dnp, restoring the full fluorescence yield of the donor. The increase in fluorescence is linearly related to the rate of proteolysis.

Briefly in a 384-well format recombinant BACE2 protein in a final concentration of 0.4 μg/ml is incubated for 450 minutes at room temperature with 10 μM substrate in incubation buffer (50 mM Citrate buffer pH 5.0, 0.05% PEG, no DMSO) in the absence or presence of compound. Next the amount of proteolysis is directly measured by fluorescence measurement at T=0 and T=450 (excitation at 320 nm and emission at 405 nm). Results are expressed in RFU (Relative Fluorescence Units), as difference between T450 and TO.

A best-fit curve is fitted by a minimum sum of squares method to the plot of % Controlmin versus compound concentration. From this an IC₅₀ value (inhibitory concentration causing 50% inhibition of activity) can be obtained.

$$LC = Median of the low control values = Low control \text{:} Reaction without enzyme$$

$$HC = Median of the High control values = High control \text{:} Reaction with enzyme$$

$$\% Effect = 100 \text{-} [ ( sample \text{-} LC ) / ( HC \text{-} LC ) * 100 ]$$

$$\% Control = ( sample / HC ) * 100$$

$$\% Controlmin = ( sample \text{-} LC ) / ( HC \text{-} LC ) * 100$$

The following exemplified compounds were tested essentially as described above and exhibited the following the activity:

TABLE 8

| | Biochemical FRET |
|---|---|
| Co. No. | based assay pIC₅₀ |
| 1 | 8.18 |
| 2 | 7.36 |
| 5 | 7.1 |
| 9 | 6.56 |

n.t. means not tested

### Biochemical Assay—Automated

### General Methods

Unless otherwise indicated all biochemicals were purchased from Sigma-Aldrich Chemical Company, Poole, Dorset, U.K. and non-aqueous solvents, of analytical or higher grade, were purchased from ThermoFisher Scientific, Loughborough, U.K. MilliQ water (Elix 5 & MilliQ Gradient; Merck Millipore) was used as the base aqueous solvent to make up the biological buffers. Base assay buffer was prepared by adding a 50 mM solution of citric acid (1.00244; Merck Biosciences) to stirring solution of 50 mM trisodium citrate (1.06448; Merck Biosciences) until a final pH of 5.0 was achieved. To this was added a 40% solution of polyethylene glycol (“PEG”) (P1458; Sigma Aldrich) to a final concentration of 0.05%; hence base buffer comprised of 50 mM sodium citrate, pH 5.0 containing 0.05% PEG. All assays were routinely carried out in 384-well assay plates (Costar 4514; Corning Life Sciences) and incubated at 37±1° C. for 60 min. prior to reading the endpoint fluorescence intensity. The (7-methoxyl coumarin-4-yl)acetic acid based substrate 0-secretase substrate VI (M2465; Bachem) was prepared as a 1 mM stock in 100% DMSO (D/4121/PB08; ThermoFisher). Assay buffer was prepared by adding DMSO to base buffer to a final concentration of 1% (vol./vol.). β-secretase I (18.64 μM; “BACE1”) and β-secretase II (4.65 μM; “BACE2”) were obtained from Janssen Pharmaceutica, Beerse, Belgium and were stored as frozen aliquots (˜20 μl) and thawed as required.

### Manual Assays

Typically 12.5 μl of assay buffer was dispensed to rows B to P of the assay plate. To row A was added 18.75 μl of test compound diluted appropriately in assay buffer. A 6.25 μl aliquot of sample was transferred from row A to row B and the sample mixed three times by pipette. The process was repeated down the plate and 6.25 μl of solution discarded at row N post-mix. Rows O and P were designated as the positive and negative controls. To row P was added 6.25 μl base buffer. To rows A to 0 was added 6.25 μl enzyme (freshly prepared 40 nM BACE1 or 40 nM BACE2) diluted in base buffer. To initiate the assay 6.25 μl of freshly prepared 80 μM substrate, made up by diluting the 1 mM in 100% DMSO solution into HPLC grade water (Optima W6-212; ThermoFisher), was added to all the wells. The assay plate was covered and incubated at 37±1° C. for 60 min. The fluorescence intensity of the wells was read at 360/405 nm (excitation/emission) utilising a nine reads per well protocol (50 ms integration; density of 3, 0.25 mm spacing; SpectraMAX Paradigm plate reader; TUNE cartridge; SoftMax Pro v 6.3 software; Molecular Devices UK Ltd., Wokingham, Berkshire, UK) and outputting the median value of the nine reads as a text file. Data analysis was carried out using Prism software v 6.3 (GraphPad Inc., San Diego, Calif., USA) using the non-linear regression analysis models supplied by the vendor. For IC₅₀ determinations the four parameter logistic variable slope model was used to fit the raw fluorescence intensity data with the ‘bottom’ fixed to the negative control.

### Automated Bioassay Hardware

The CyclOps bioassay module consisted of a fraction collection station, a reagent station, liquid handling robotics, plate store and an integrated plate reader (SpectraMAX Paradigm, TUNE cartridge, SoftMax Pro v 6.3; Molecular Devices). The fraction collection station composed of a 384 well collection plate (P-384-240SQ-C; Axygen, Union City, Calif., USA) mounted on a H-portal carriage (Festo AG & Co. KG, Esslingen, Germany), a syringe drive and a two-way six port injection valve fitted with a 200 μl loop (VICI AG International, Schenkon Switzerland). The output of the injection valve was addressable to all the positions of a 384 well collection plate. The reagent station consisted of hydraulically cooled (10-12° C.) aluminium segments; each manufactured to house a SBS microtiter plate footprint. Independent addressable reagent stations were housed within these sections. Where required, custom aluminium housings were used to accommodate standard laboratory plastic ware (e.g. Eppendorf tubes, Falcon tubes, etc.). As and when required the reagent reservoirs were covered and the lids contained holes through which the Teflon-coated probe could access solutions. The reagents present on the liquid handling system were:
Probe wash solution (˜150 ml; 33.3:33.3:33.3 water:propan-2-ol (P/7508/17; ThermoFisher):methanol (M/4058/17; ThermoFisher) contained in a covered reagent reservoir (390007; Porvair Sciences Ltd., Leatherhead, UK).
Assay buffer solution
HPLC grade water
40 nM BACE1 diluted in base buffer contained in a 5 ml Eppendorf tube (0030 119.401; Eppendorf)
400 nM BACE2 diluted in 25 mM tris (648311; Merck Biosciences, Nottingham, U.K.), pH 7.5 containing 100 mM sodium chloride and 20% glycerol (16374; USB Corp., Cleveland, Ohio, USA) contained in a 1.5 ml Eppendorf tube (0030 000.919; Eppendorf)
1 mM substrate in 100% DMSO contained in a 1.5 ml Eppendorf tube (maintained at ambient temperature)
Two empty 1.5 ml Eppendorf tubes

The liquid handling system composed of a LISSY system (Zinsser Analytik GmbH, Frankfurt, Germany) equipped with gripper arm and single teflon-coated stainless steel probe. Between every liquid handling step the teflon-coated stainless steel probe was washed with probe wash solution followed by system liquid (water). Control of the bioassay system was achieved using WinLISSY software (Zinsser Analytik) and SoftMax Pro (which was under WinLISSY automation command control). A plate store housed a stack of assay plates (Costar 4514). Input and output relays enabled contact closure control and feedback between the bioassay module and the CyclOps control software. The plate store was an aluminium rack that accommodated a stack of assay plates which could be accessed by the liquid handling system.

### The Automated Bioassay Process

The output of the dilution module flowed through the collection station injection valve set in the ‘load’ position. With WinLISSY set to input polling mode contact closure by the CyclOps control software initiated the bioassay protocol. The first action triggered the injection valve to the ‘inject’ position, isolating the loop contents, and the fraction collection system dispensed the loop contents to an addressable well on the collection plate. Concomitantly the liquid handling system delivered an assay plate to an assay station on the liquid handling bed. Onto columns of the assay plate the liquid handling system dispensed 12.5 μl assay buffer down two columns of the assay plate from row B to row P. To row A was added 18.75 μl of test compound from the respective well of the collection plate. A 6.25 μl aliquot of sample from row A was transferred to row B. The process was repeated down the plate for both columns and 6.25 μl reagent discarded at row N. Rows 0 and P were designated as the positive and negative controls. To row P was added 6.25 μl assay buffer. To rows A to 0 of the first column was added 6.25 μl 40 nM BACE1 stored in base buffer. For the BACE2 enzyme addition, 17.5 μl of 400 nM BACE2 was diluted with 157.5 μl base buffer. This was mixed by pipetting 175 μl of solution five times in the designated receiving Eppendorf tube and then 6.25 μl of the diluted BACE2 was added up the respective column. For the MCA substrate, 30.8 μl of 1 mM MCA substrate in 100% DMSO was diluted with 385 μl HPLC water. This was mixed by pipetting 400 μl five times in the designated receiving Eppendorf tube and 6.25 μl added up the respective columns. The assay plate was then transferred to the plate reader carriage, the drawer closed and the assay incubation initiated. After 60 min. WinLISSY executed a sub-routine that instructed the plate reader to load and execute a protocol file which read the fluorescence intensity. This protocol file contained the parameters required to read the microtiter plate and write the corresponding data as a text file. Fluorescence intensity was read at 360/405 nm (excitation/emission) utilising a nine reads per well protocol (50 ms integration; density of 3, 0.25 mm spacing) and outputted the median value of the nine reads as a text file.

### CyclOps Bioassay Data Analysis

CyclOps software was set to poll the bioassay shared data file folder. On saving the data, WinLISSY sent an output contact closure signal notifying the CyclOps software that the bioassay had been completed. CyclOps software opened, processed and analysed the data. Data processing consisted of appending the respective concentration of test article to the corresponding rows (with data received from the dilution module). Thereafter the data was analysed (MATLAB; MathWorks, Cambridge, U.K.) by a non-linear regression analysis employing a four parameter logistic model to determine the IC₅₀. The span was fixed between baseline (i.e. row P) and the maximum observed positive control rate (i.e. row 0). To maintain data quality, rules were set up to govern automated bioassay data analysis. In the first instance if no less than seventy-five percent activity or no greater than twenty-five percent activity were observed the data was rejected. This ensured that there was sufficient titration data for good analysis to be carried out. Thereafter the quality of the fit was judged by the R-squared value. If this value fell below 0.85 then the data was rejected. In all cases rejection led to a bioassay failure tag being reported to the system. Outlier analysis was carried out as described previously (Motulsky, H. J. and Brown, R. E., (2006), BMC Bioinformatics, 7, 123) with a Q value of 10%. For the automated IC₅₀ analysis a maximum of three outliers could be excluded prior to an error of fit flag being generated. Cross validation of the bioassay data was achieved by analysing the same using Prism software v 6.3 (GraphPad Inc.) employing a non-linear regression analysis four parameter logistic variable slope model to fit the raw fluorescence intensity data with the ‘bottom’ fixed to the negative control.

TABLE 9

| | Automated assay | Automated assay |
|---|---|---|
| Co. No. | BACE1 pIC₅₀ | BACE2 pIC₅₀ |
| 3 | 7.64 | 6.45 |
| 5 | 7.31 | 6.69 |
| 6 | 7.12 | 6.72 |
| 7 | 7.21 | 6.96 |
| 8 | 6.71 | 6.66 |

1. A compound of Formula (I) 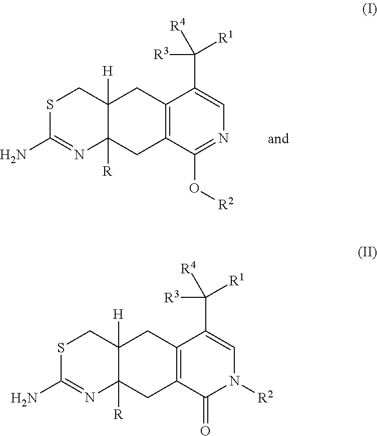
or a tautomer or a stereoisomeric form thereof, wherein
R is phenyl optionally substituted with 1, 2, or 3 substituents each independently selected from the group consisting of halo, C₁₋₃ alkyloxy, cyano, 2-cyano-pyridin-5-yl, 3-cyano-pyridin-5-yl, and pyrimidin-5-yl;
R¹ is selected from the group consisting of C₁₋₃ alkyl; C₃₋₆cycloalkyl optionally substituted with C₁₋₃alkyl; aryl; heteroaryl; and 4-tetrahydro-2H-pyranyl optionally substituted with C₁₋₃alkyl; with the provisos that
a) R¹ is C₃₋₆cycloalkyl optionally substituted with C₁₋₃alkyl; aryl; heteroaryl; or 4-tetrahydro-2H-pyranyl optionally substituted with C₁₋₃alkyl; when R³ is hydrogen and R⁴ is hydrogen or C₁₋₃alkyl; or
b) R¹ is C₁₋₃alkyl; C₃₋₆cycloalkyl optionally substituted with C₁₋₃alkyl; aryl; heteroaryl; or 4-tetrahydro-2H-pyranyl optionally substituted with C₁₋₃alkyl; when R³ is hydrogen and R⁴ is C₁₋₃alkyloxy; or
c) R¹ is C₃₋₆cycloalkyl optionally substituted with C₁₋₃alkyl; or 4-tetrahydro-2H-pyranyl optionally substituted with C₁₋₃alkyl; when R³ is hydrogen and R⁴ is C₃₋₆cycloalkyl; or
d) R¹ is C₁₋₃alkyl; C₃₋₆cycloalkyl optionally substituted with C₁₋₃alkyl; aryl; heteroaryl; or 4-tetrahydro-2H-pyranyl optionally substituted with C₁₋₃alkyl; when >CR³R⁴ is >(C═O); wherein
aryl is phenyl or phenyl substituted with 1, 2 or 3 substituents each independently selected from the group consisting of halo, cyano, C₁₋₃alkyl, mono-halo-C₁₋₃alkyl, poly-halo-C₁₋₃alkyl, C₃₋₆cycloalkyl, C₁₋₃alkyloxy, mono-halo-C₁₋₃alkyloxy and polyhalo-C₁₋₃alkyloxy;
heteroaryl is selected from the group consisting of pyridyl, pyrimidyl, pyrazinyl, pyridazinyl, furanyl, thienyl, pyrrolyl, pyrazolyl, imidazolyl, triazolyl, tetrazolyl, thiazolyl, isothiazolyl, thiadiazolyl, oxazolyl, isoxazolyl, oxadiazolyl, indolyl, indazolyl, 1H-benzimidazolyl, benzoxazolyl, and benzothiazolyl, each of which being optionally substituted with 1, 2, or 3 substituents, each independently selected from the group consisting of halo, cyano, C₁₋₃alkyl, mono-halo-C₁₋₃alkyl, poly-halo-C₁₋₃alkyl, C₃₋₆cycloalkyl, C₁₋₃alkyloxy, mono-halo-C₁₋₃alkyloxy and polyhalo-C₁₋₃alkyloxy;
R² is hydrogen or C₁₋₃alkyl;
R³ is hydrogen; and
R⁴ is hydrogen or C₁₋₃alkyl; or
R³ and R⁴ taken together are C═O;
or a pharmaceutically acceptable addition salt or a solvate thereof.

2. The compound according to claim 1, wherein
R¹ is C₃₋₆cycloalkyl optionally substituted with C₁₋₃alkyl; aryl; heteroaryl; or
4-tetrahydro-2H-pyranyl optionally substituted with C₁₋₃alkyl;
wherein
aryl is phenyl or phenyl substituted with 1, 2 or 3 substituents each independently selected from the group consisting of halo, cyano, C₁₋₃alkyl, mono-halo-C₁₋₃alkyl, poly-halo-C₁₋₃alkyl, C₃₋₆cycloalkyl, C₁₋₃alkyloxy, mono-halo-C₁₋₃alkyloxy and polyhalo-C₁₋₃alkyloxy; and
heteroaryl is selected from the group consisting of pyridyl, pyrimidyl, pyrazinyl, pyridazinyl, furanyl, thienyl, pyrrolyl, pyrazolyl, imidazolyl, triazolyl, tetrazolyl, thiazolyl, isothiazolyl, thiadiazolyl, oxazolyl, isoxazolyl, oxadiazolyl, indolyl, indazolyl, 1H-benzimidazolyl, benzoxazolyl, and benzothiazolyl, each of which being optionally substituted with 1, 2, or 3 substituents, each independently selected from the group consisting of halo, cyano, C₁₋₃alkyl, mono-halo-C₁₋₃alkyl, poly-halo-C₁₋₃alkyl, C₃₋₆cycloalkyl, C₁₋₃alkyloxy, mono-halo-C₁₋₃alkyloxy and polyhalo-C₁₋₃alkyloxy;
R³ is hydrogen; and
R⁴ is hydrogen or C₁₋₃alkyl.

3. The compound according to claim 1 or 2, wherein
aryl is phenyl or phenyl substituted with 1, 2 or 3 substituents each independently selected from the group consisting of of halo, cyano, C₁₋₃alkyl, mono-halo-C₁₋₃alkyl, poly-halo-C₁₋₃alkyl, C₃₋₆cycloalkyl, and C₁₋₃alkyloxy; and
heteroaryl is selected from the group consisting of pyridyl, pyrimidyl, pyrazinyl, pyridazinyl, furanyl, thienyl, pyrrolyl, pyrazolyl, imidazolyl, triazolyl, tetrazolyl, thiazolyl, isothiazolyl, thiadiazolyl, oxazolyl, isoxazolyl, and oxadiazolyl, each of which being optionally substituted with 1, 2, or 3 substituents, each independently selected from the group consisting of halo, cyano, C₁₋₃alkyl, mono-halo-C₁₋₃alkyl, poly-halo-C₁₋₃alkyl, C₃₋₆cycloalkyl, and C₁₋₃alkyloxy.

4. The compound according to claim 1, wherein
R¹ is aryl; heteroaryl; or 4-tetrahydro-2H-pyranyl optionally substituted with C₁₋₃alkyl;
aryl is phenyl or phenyl substituted with 1, 2 or 3 substituents each independently selected from the group consisting of of halo, C₁₋₃alkyl, and
C₁₋₃alkyloxy; and
heteroaryl is selected from the group consisting of pyridyl, pyrimidyl, pyrazinyl, pyridazinyl, furanyl, thienyl, pyrrolyl, pyrazolyl, imidazolyl, triazolyl, tetrazolyl, thiazolyl, isothiazolyl, thiadiazolyl, oxazolyl, isoxazolyl, and oxadiazolyl, each of which being optionally substituted with 1, 2, or 3 substituents, each independently selected from the group consisting of halo,
C₁₋₃alkyl, and C₁₋₃alkyloxy; and
R³ and R⁴ are each hydrogen.

5. The compound according to claim 1, wherein R is phenyl optionally substituted with 1, or 2 independently selected halo substituents.

6. The compound according to claim 1, wherein R² is C₁₋₃alkyl.

7. A pharmaceutical composition comprising a therapeutically effective amount of a compound according to claim 1 and a pharmaceutically acceptable carrier.

8. A process for preparing a pharmaceutical composition comprising mixing a pharmaceutically acceptable carrier with a therapeutically effective amount of a compound according to claim 1.

9. (canceled)

10. (canceled)

11. A method of treating a disorder selected from the group consisting of Alzheimer's disease, mild cognitive impairment, senility, dementia, dementia with Lewy bodies, Down's syndrome, dementia associated with stroke, dementia associated with Parkinson's disease, and dementia associated with beta-amyloid comprising administering to a subject in need thereof, a therapeutically effective amount of a compound according to claim 1.

12. A method for modulating beta-site amyloid cleaving enzyme activity, comprising administering to a subject in need thereof, a therapeutically effective amount of a compound according to claim 1.

13. (canceled)

14. A process for the preparation of a compound according to Formula (I) and (II) wherein R, R¹, R², R³ and R⁴ are as defined in any one of claims 1 to 6, comprising subjecting a compound of Formula (III) or (IV) wherein X represents halo or triflate, to a Negishi type reaction, with an organozinc compound of Formula (V) wherein X′ is halo and R¹, R³ and R⁴ are as defined in claim 1, in the presence of a Palladium(0) species, as represented in steps a) or b) 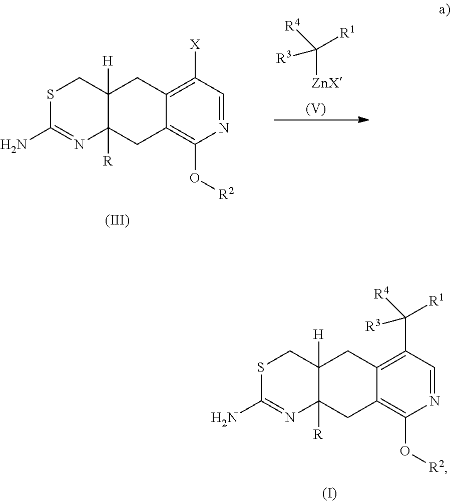
or

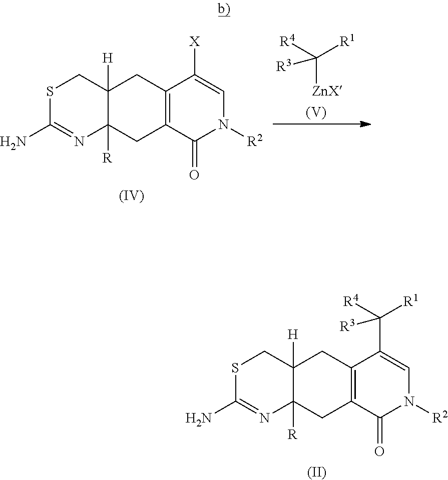

15. A compound of Formula (III) or (IV), wherein R, R¹, R², R³ and R⁴ are as defined in any claim 1, and X is halo

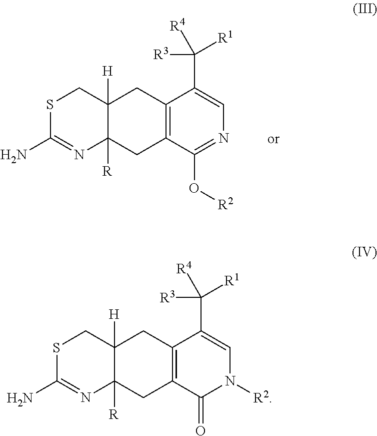

16. The compound according to claim 6, wherein R² is methyl.

17. A method of treating a disorder selected from the group consisting of Alzheimer's disease, mild cognitive impairment, senility, dementia, dementia with Lewy bodies, Down's syndrome, dementia associated with stroke, dementia associated with Parkinson's disease, and dementia associated with beta-amyloid comprising administering to a subject in need thereof, a therapeutically effective amount of a pharmaceutical composition according to claim 7.

18. A method for modulating beta-site amyloid cleaving enzyme activity, comprising administering to a subject in need thereof, a therapeutically effective amount of a pharmaceutical composition according to claim 7.
